(19) United States
(12) Patent Application Publication
(10) Pub. No.: US 2019/0292549 A1
(43) Pub. Date: Sep. 26, 2019

(54) POLY(ETHYLENE GLYCOL) BRUSHES FOR EFFICIENT RNA DELIVERY

(71) Applicant: NORTHEASTERN UNIVERSITY, Boston, MA (US)

(72) Inventors: Ke ZHANG, Boston, MA (US); Dali Wang, Boston, MA (US)

(21) Appl. No.: 16/253,547

(22) Filed: Jan. 22, 2019

Related U.S. Application Data
(60) Provisional application No. 62/646,542, filed on Mar. 22, 2018.

(51) Int. Cl. C12N 15/113; C12N 15/87; A61K 47/10; A61K 47/69; A61K 47/56

(52) U.S. Cl. CPC C12N 15/1136; C12N 15/87; C12N 2310/141; A61K 47/69; A61K 47/56; A61K 47/10

(57) ABSTRACT

Disclosed are bottlebrush poly(ethylene glycol) (PEG) polymer-RNA conjugates (pacRNA) having a plurality of PEG side chains attached to a polymer backbone, and one or more RNA oligonucleotides attached to the polymer backbone via a cleavable linkage. The pacRNAs offer potential useful novel RNA-based therapeutics.

### CROSS REFERENCE TO RELATED APPLICATIONS

This application claims the benefit of priority under 35 U.S.C. § 119 to U.S. Provisional Patent Application No. 62/646,542 by Zhang and Wang, filed Mar. 22, 2018, the entire disclosure of which is incorporated herein by reference thereto.

### STATEMENT REGARDING FEDERALLY SPONSORED RESEARCH OR DEVELOPMENT

This invention was made with government support under grant number 1453255 awarded by the National Science Foundation. The government has certain rights in the invention.

### FIELD OF THE INVENTION

This application relates to brush polymer-RNA oligonucleotide conjugates comprising RNA oligonucleotides covalently attached to the backbone of a sterically congested PEG brush polymer and the use of such polymer-oligonucleotide conjugates in siRNA gene regulation.

### BACKGROUND OF THE INVENTION

Small-interfering RNAs (siRNAs) represent a new therapeutics paradigm for treating various diseases including viral infections, hereditary disorders, and cancers. siRNA therapeutics offer promising alternatives to small molecule inhibitors, especially for “undruggable” targets. Compared to antisense oligonucleotides (ASOs), RNA interference (RNAi) is believed to be a more efficient and robust technology for gene suppression, owing to the involvement of the catalytic RNA-induced silencing complex (RISC). Considerable amounts of effort and capital have been invested in bringing siRNA therapeutics to the market. To date, at least 20 RNAi-based drug candidates have entered clinical trials worldwide, with several GaINAc-siRNA conjugates in late-stage trials in the U.S. A lipid particle-formulated siRNA, patisiran, was recently approved by the U.S. Food and Drug Administration for the treatment of polyneuropathy caused by hereditary transthyretin amyloidosis, becoming the long-awaited breakthrough in RNA therapeutics (D. Adams et al., Patisiran, an RNAi therapeutic, for hereditary transthyretin amyloidosis, N. Engl. J. Med., 379, 11-21 (2018); C. Chakraborty et al., Therapeutic miRNA and siRNA: moving from bench to clinic as next generation medicine, Mol. Ther. Nucleic Acids, 8, 132-143 (2017)). Yet, despite major progress, clinical translation is mainly limited to diseases of or originating from the liver. (C. V. Pecot et al., RNA interference in the clinic: challenges and future directions, Nat. Rev. Cancer, 11, 59-67 10 (2011); J. K. Nair et al., Multivalent N-acetylgalactosamine-conjugated siRNA localizes in hepatocytes and elicits robust RNAi-mediated gene silencing, J. Am. 14 Chem. Soc., 136, 16958-16961 (2014)). A key challenge to realizing the broad potential of siRNA-based therapeutics involves the delivery of siRNAs to non-liver organs and tissues and across the plasma membrane of cells in vivo. Unmodified siRNA is digested by serum and cellular nucleases and is subject to rapid renal clearance due to its small size, and thus has a half-life too short for clinical use. Additionally, naked siRNA does not readily enter unperturbed cells even at millimolar concentrations due to a combination of large size (^{˜}13-16 kDa), negative charge (^{˜}−35 mV), and hydrophilicity. Methods to address these challenges include advanced delivery systems (e.g., polycationic polymers, lipids, proteins, and peptides) and direct chemical modifications of the oligonucleotide. The use of cationic species for electrostatic complexation/encapsulation with siRNA is efficient in facilitating cellular uptake and promoting RNAi activity in vitro but leads to greater potential for immunogenic responses, toxicity, and inconsistency in formulation (especially for complex delivery systems with multiple components) (M. S. Shim, Y. J. Kwon, Efficient and targeted delivery of siRNA in vivo, FEBS J., 277, 34 4814-4827 (2010); J. E. Dahlman, et al., In vivo endothelial siRNA delivery using polymeric nanoparticles with low molecular weight, Nat. Nanotechnol, 9, 648-655 (2014)). Other than liposomes, carrier-based systems have not proven to be relevant in a clinical setting (C. Chakraborty et al). On the other hand, modification chemistries have greatly improved enzymatic stability, potency, and duration of RNAi in vivo, making it possible to bypass the need for complex carriers. In addition, certain modifications (e.g. phosphorothioates, PS) result in non-specific binding with serum proteins and thus avoidance of renal clearance and improved tissue uptake (F. Eckstein, Phosphorothioates, essential components of therapeutic oligonucleotides, Nucleic Acid Ther. 24, 374-387 (2014)). However, liver often remains the organ to receive the majority of the injected dose, followed by kidney, bone marrow, adipocytes, and lymph nodes. Other possible drawbacks associated with chemical modifications may include liver and cardiovascular toxicity, prolonged blood coagulation times, thrombocytopenia, and reduced binding affinity for the target sequence. Therefore, to realize the full potential of siRNA drugs, a safe, simple, and efficient vector system that can address non-liver targets, is still very much sought after.

### SUMMARY OF THE INVENTION

In one aspect of the invention, there is provided a bottlebrush poly(ethylene glycol) (PEG) polymer-RNA conjugate (pacRNA) comprising one or more RNA oligonucleotides and a PEG brush polymer comprising a plurality of PEG side chains and a polymer backbone, wherein each of the one or more RNA oligonucleotides is attached to the polymer backbone via a cleavable linkage. In one embodiment, the one or more RNA oligonucleotides are siRNA. In certain embodiments, the polymer backbone is selected from poly(norbornene), poly(styrene), poly(meth)acrylate, polypeptide, polyether, polyamide and polyurethane. In certain embodiments, the polymer backbone is poly(norbornene). In embodiments of this aspect of the invention, the PEG brush polymer has 10 to 60 repeating polymer units in the backbone. In certain embodiments, the PEG side chains are each at least 3 kDa, preferably greater than 5 kDa. In other embodiments the PEG side chains range from 5 kDa to 40 kDa. In certain embodiments, the PEG brush polymer includes from 25 to 60 side chains attached to the backbone and each side chain is at least 10 kDa. The overall molecular weight of the PEG brush polymer side chains is about 75 to 500 kDa. In a preferred embodiment, the pacRNA is designed with 30 PEG_{10 kDa} repeating units attached to a poly(norbornene) backbone. In certain embodiments of this aspect and other aspects of the invention, the one or more RNA oligonucleotides are attached to the polymer backbone by a linkage that is cleavable, such as via enzymatic, hydrolytic, or a stimuli responsive cleavage. Such stimuli responsive cleavage includes response to pH, light, UV light, ultrasound and the like. In other embodiments of the various aspects of the invention, the RNA oligonucleotides are attached to the polymer backbone by a bioreductively cleavable linkage.

In another aspect of the invention, there is provided a method of inhibiting expression of a gene product encoded by a target polynucleotide. The method comprises contacting a cell containing the target polynucleotide with a bottlebrush poly(ethylene glycol) (PEG) polymer-RNA conjugate (pacRNA) comprising one or more RNA oligonucleotides and a PEG brush polymer comprising a plurality of PEG side chains and a polymer backbone to obtain uptake of the pacRNA by the cell, wherein the one or more RNA oligonucleotides are attached to the polymer backbone via a cleavable linkage and wherein the one or more RNA oligonucleotides have a sequence that is complementary to at least a portion of the target polynucleotide. In one embodiment of this aspect of the invention, the one or more RNA oligonucleotides are siRNA. In certain embodiments of this aspect of the invention, the cell is a cancer cell.

In another aspect of the invention, there is provided a method for promoting cellular uptake of an RNA oligonucleotide. The method comprises contacting a pacRNA structure with a cell, wherein the pacRNA comprises one or more RNA oligonucleotides and a PEG brush polymer comprising a plurality of PEG side chains and a polymer backbone, wherein the one or more RNA oligonucleotides are attached to the PEG backbone via a cleavable linkage, such as a bioreductively cleavable linkage.

In yet another aspect of the invention, there is provided a composition comprising a bottlebrush poly(ethylene glycol) (PEG) polymer-RNA conjugate (pacRNA) comprising one or more RNA oligonucleotides and a PEG brush polymer comprising a plurality of PEG side chains and a polymer backbone, wherein each of the one or more RNA oligonucleotides is attached to the polymer backbone via a cleavable linkage, such as a bioreductively cleavable linkage.

In another aspect of the invention, there is provided a kit for inhibiting gene expression of a target polynucleotide. In one embodiment of this aspect, the kit contains at least one type of bottlebrush poly(ethylene glycol) (PEG) polymer-RNA conjugate (pacRNA) comprising one or more RNA oligonucleotides and a PEG brush polymer comprising a plurality of PEG side chains and a polymer backbone, wherein each of the one or more RNA oligonucleotides is attached to the polymer backbone via a cleavable linkage and wherein each of the RNA oligonucleotides is complementary to at least a portion of the target polynucleotide. In one embodiment of this aspect of the invention, the kit contains a first type of pacRNA having one or more RNA oligonucleotides having a sequence complementary to one or more sequences of a first portion of a target polynucleotide. The kit optionally includes one or more additional types of pacRNA which have a sequence complementary to a second portion of the target polynucleotide or to a second target sequence.

### BRIEF DESCRIPTION OF THE DRAWINGS

FIG. 1 is a schematic showing the synthetic route for pacRNA and pac-SS-RNAs (pacRNA_{Nclv} and pacRNA_{Clv}).

FIG. 2a-2e shows physical characterization of pacRNA. 2a is the chemical structure of pacRNA.

FIG. 2b is a coarse-grained molecular dynamics simulation of the pacRNA (1 μs simulation with explicit water using the MARTINI force field). A crystal structure of E. coli RNase III is placed next to the padRNA for size comparison. FIG. 2c is an aqueous GPC chromatogram and agarose gel electrophoresis (1%, inset) of pacRNA and free siRNA. FIG. 2d shows a DLS intensity-average size distribution of pacRNA_{Clv}. The inset shows ζ-potential measurements of siRNA and pacRNAs in Nanopure™ water. FIG. 2e is a TEM image of pacRNA_{Clv}, negatively stained with 2% uranyl acetate.

FIG. 3a-d shows reductive release, hybridization, and enzymatic degradation of pacRNA. FIG. 3a is an agarose gel electrophoresis showing the reductive release of siRNA from pacRNA_{Clv} in the resence 10 mM DTT as a function of time; release profile by gel band densitometry analysis is shown to the right. FIG. 3b shows schematics of enzymatic digestion kinetics assay based upon fluorophore- and quencher-tagged RNA. FIGS. 3c and 3d show RNA hybridization and RNase III degradation kinetics for pacRNAs, 40 kDa Y-shaped PEG-RNA conjugate, and free RNA.

FIG. 4a-f shows In vitro cellular uptake and gene silencing using pacRNA. 4a is a flow cytometry measurement (total cell count: 10,000) of SKOV3 cells treated with PO RNA, PS RNA, and pacRNA (100-2000 nM) for 4 h. 4b shows the mean fluorescent intensity of cells treated with free RNA (PS and PO) and pacRNACIv. 4c shows confocal microscopy images showing intracellular GSH-triggered release of siRNA from pacRNACIv in high-GSH cells (SKOV3 and SKBR3). Fluorescence is turned on when the fluorophore (fluorescein)-labeled siRNA is released from quencher (dabcyl)-labeled brush polymer. Controls include low-GSH HDF cells (negative) and cells pretreated with 10 mM GSH-OEt (positive). d, qRT-PCR measurement (mean±SD, n=3) of Bcl-2 transcript levels in SKOV3 cells treated with pacRNAs, free siRNA, and pacRNACIv containing a scrambled control sequence. 4e is Bcl-2 protein levels characterized by western blotting. 4f shows cell apoptosis following sample treatment determined by annexin V and PI staining. Early apoptotic, late apoptotic, and necrotic cell populations (%) are shown in the lower right, upper right, and upper left quadrants, respectively. Results are representatives of three independent flow cytometry measurements. **P<0.01 (two-tailed t-test).

FIG. 5a-c shows cellular uptake of PO siRNA, PS RNA, and pacRNA in SKOV3 cells. 5a is a representative confocal image of SKOV3 cells treated with Cy3-labled PO siRNA and pacRNA (red) for 4 h. Cell nuclei were stained with Hoechst 33342 (blue). Imaging setting were kept identical in (a). 5b shows flow cytometry measurements (total cell counts: 10,000) of SKOV3 cells treated with varying concentrations of ds PS RNA for 4 h. 5c are representative confocal images of SKOV3 cells treated with Cy3-labled ss PS RNA and ds PS RNA for 4 h. Imaging setting were kept identical in (c). Scale bar: 20 μm.

FIG. 6a-c shows the cellular uptake of PO siRNA, PS RNA (ss and ds), and pacRNA in SKBR3 cells. 6a shows flow cytometry measurements (total cell count: 10,000) of SKBR3 cells treated with varying concentrations of samples (100-2000 nM RNA) for 4 h. FIG. 6b is a combined analysis of flow cytometry mean fluorescence showing the different rates of SKBR3 cell uptake of PO siRNA, PS RNA (ss and ds), and pacRNA_{Clv}. 6c are representative confocal images of SKBR3 cells treated with Cy3-labled PO siRNA and pacRNAs (red) for 4 h. Cell nuclei were stained with Hoechst 33342 (blue). Imaging setting were kept identical across samples. Scale bar: 20 μm. Error bars refer to the standard deviation of the mean of three independent measurements.

FIG. 7 shows representative confocal images of SKBR3 cells treated with Cy3-labled ss PS RNA or ds PS RNA (red) for 4 h. Cell nuclei were stained with Hoechst 33342 (blue). Scale bar: 20 μm. Imaging setting were kept identical across samples.

FIG. 8a-d shows Bcl-2 downregulation and cell apoptosis induced by pacRNA. (a, b) Relative Bcl-2 levels determined by band densitometry analysis of western blots. Bcl-2 expression in SKOV3 and SKBR3 cells were treated with pacRNA and controls (1 μM siRNA) for 6 h in serum-free media, followed by an additional 66 h incubation in fresh, full-serum media. (c) A representative western blot of SKBR3 cells treated with pacRNA and controls. (d) Apoptotic cell populations following sample treatment as determined by flow cytometry. Error bars refer to the standard deviation of three independent measurements.

FIG. 9a-c shows the cytotoxicity and blood compatibility of pacRNA. 9a is a graph of the cell viability of SKOV3 cells treated with pacRNAs and controls. 9b shows hemolysis of human blood (type O+) treated with pacRNAs and controls, as determined by spectrophotometric measurement of hemoglobin present in the supernatant of centrifuged RBC suspensions. The % RBC hemolysis is defined as the percentage of hemoglobin present in the supernatant compared with the total hemoglobin released by Triton X-100 treatment. Inset: photograph of centrifuged RBC suspensions. 9c is a graph of activated partial thromboplastin times of plasma treated with pacRNA or ss PS RNA at equal RNA concentrations. ***P<0.001 (two-tailed t-test).

FIG. 10 a-k shows pharmacokinetics, biodistribution, efficacy, and safety assessment in vivo. 10a is a graph of plasma PK of PO RNA, PS RNA, pacRNA, and brush polymer in C57BL/6 mice. 10b is fluorescence imaging of BALB/c-nu mice bearing a human ovarian SKOV3 xenograft following intravenous injection of Cy5-labeled siRNA and pacRNAs, or Cy5.5-labeled brush polymer. The circle indicates the location of tumor. 10c is ex vivo imaging of tumors and other major organs 24 h post injection. 10d shows the biodistribution determined by quantitative analysis of the fluorescence signals f ex vivo tumors and major organs. 10e is a graph of the tumor volume changes in the course of 32 days with intravenous administration of PBS, pacRNA_{Clv}, and pacRNA_{NClv} at equivalent siRNA doses every fourth day (treatment started on day 0). 10f is a graph of body weight changes of tumor-bearing mice during the course of treatment. 10g is a western blot of Bcl-2 in homogenized tumor tissues 96 h after the last treatment. 10h-j are the results of histological studies showing reduced Bcl-2 expression (immunohistochemistry staining, IHC). 10h shows increased cell apoptosis (TUNEL); 10i shows histologic apoptosis hallmarks H&E staining). 10j shows SKOV3 tumors treated with pacRNA_{Clv} compared with control groups. 10k shows cytokine levels (TNF-α, IL-6, and IL-12) in the serum in C57BL/6 mice after 8 h of treatment with pacRNAs and controls. **P<0.01, ***P<0.001 (two-tailed t-test).

FIG. 11 shows microscopic images of H&E-stained sections of various organs from mice after a 32-day treatment period with pacRNAs and PBS showing no apparent histological anomalies.

### DETAILED DESCRIPTION OF THE INVENTION

Delivery of short interfering RNAs (siRNAs) remains a key challenge in the development of RNA interference therapeutics. siRNA is a class of double-stranded RNA molecules, generally about 20-25 base pairs in that operates within the RNA interference pathway. siRNAs have a well-defined structure: a short (usually 20 to 25-bp) double-stranded RNA (dsRNA) with phosphorylated 5′ ends and hydroxylated 3′ ends with two overhanging nucleotides. An ideal siRNA vector for in vivo use must fulfill at least two main functions safely. First, it must facilitate cellular uptake and enhance RNAi activity. Second, it must direct the siRNA toward the target tissue effectively.

Herein, we report that a brush form of the noncationic biocompatible polymer, poly(ethylene glycol), can be used as a transfection vector by forming a polymer-RNA conjugate via a cleavable linkage, such as a bioreductively cleavable linkage. The densely packed PEG brush provides the embedded RNA strands with enhanced nuclease stability and improves their cell uptake. Owing to the cleavable linkage, the RNA is liberated from the brush polymer under the reducing environment of tumor cells, for example, but remains conjugated under normal physiological conditions. Remarkably, the brush polymer-mediated delivery of RNA leads to significant knockdown of the target gene without eliciting significant cytotoxicity.

As used herein, the term “PEG brush polymer” or “PEG bottlebrush polymer” means a PEG polymer having an array of macromolecular PEG polymer chains attached to a polymer backbone in sufficient proximity so that the unperturbed solution dimensions of the chains are altered. The close proximity causes overlap of adjacent chains and thus significantly alters the conformational dimensions of individual polymer chains such that they extend or alter their normal radius of gyration to avoid unfavorable interactions.

The disclosed brush polymer-RNA conjugate facilitates cellular uptake by masking the charge of the RNA, thereby moderately increasing cellular uptake. Minimizing cellular interactions and internalization allows targeting ligands to be incorporated to enhance specific cell targeting and decreases the opportunity for clearance by mononuclear phagocytes.

In one aspect of the invention, there is provided a bottlebrush poly(ethylene glycol) polymer-RNA conjugate, referred to herein as “pacRNA” (polymer assisted compaction of RNA). Bottlebrush polymers are comb-like molecules with a high density of side chains grafted along a central polymer backbone, such as a poly(norbornene) backbone or other biocompatible polymer backbone, preferably a non-cationic biocompatible polymer. The spatial congestion of the bottlebrush polymer resulting from densely spaced PEG side chains provides steric shielding for the RNA much more effectively than linear or slightly branched PEG, which circumvents many of the side effects associated with specific and non-specific nucleic acid-protein interactions. Lacking these interactions, the pacRNAs exhibit considerably prolonged blood circulation times (^{˜}25×higher elimination half-life compared with unmodified siRNA) and elevated drug plasma availability (^{˜}19×greater area under the curve, AUCₒₒ), which provides an enhanced target cell permeability and retention (EPR) effect. The stability of pacRNA makes it more likely to survive the digestive endosomal environment and successfully deliver at least a fraction of the intact conjugate to the cytosol, where release of siRNA triggers RNAi.

The bottlebrush architecture of the pacRNAs also assists in realizing the second major purpose of a vector by substantially increasing the molecular weight of the siRNA (or other RNA oligonucleotide). The large size allows the siRNA to effectively escape the glomerular filtration without needing to bind with serum proteins, which enables prolonged blood retention and pronounced passive targeting of tumor tissues, for example, via the EPR (enhanced permeability and retention) effect. In addition, the steric shielding limits potential specific/non-specific interactions between the siRNA and blood, cell membrane, and intracellular proteins, which should reduce the likelihood for side effects including coagulopathy and inadvertent activation of the immune system. Past efforts to PEGylate oligonucleotides failed to achieve these substantial improvements in biopharmaceutical properties because of the use of mainly linear or slightly branched PEG, which result in insufficient local density surrounding the oligonucleotide payload. Importantly, all observed physiochemical and biopharmaceutical enhancements over naked siRNA are realized using primarily PEG (other than the brush backbone), which should promote the overall safety of the vector.

Being a molecular entity with a well-defined structure as opposed to a supramolecular assembly of various components is another advantage of the pacRNA over heterogeneous vectors, which pose additional challenges in large-scale manufacturing and batch-to-batch consistency. Thus, PEG, a biologically benign polymer normally lacking vector-like properties, has been transformed into an efficient in vivo vector by altering its architecture and coupling the structure with siRNA.

Preparation and Physicochemical Characterization of pacRNA

The brush polymer component of the pacRNA may be a homopolymer, di-block copolymer, a tri-block copolymer, etc., e.g., where one or more polymer blocks, such as poly(norbornene), polystyrene, poly(meth)acrylate, polypeptide, polyether, polyamide, polyurethane, or other polymer, are attached with RNA oligonucleotides and PEG block(s) form the sidechains attached to the polymer backbone. All of the polymer blocks together form the backbone of the PEG brush polymer. The brush polymer component of the pacRNA consists of a polymer backbone, preferably a biocompatible polymer backbone and a plurality of PEG side chains tethered to the backbone in close proximity.

For optimal shielding of the siRNA and avoidance of renal clearance, the brush polymer has PEG side chains that are sufficiently dense and long and have an overall molecular weight above the renal filtration limit. In certain embodiments of this aspect of the invention, the PEG brush polymer includes from 10 to 60 repeating polymer units in the backbone, more preferably from 20 to 50 repeating units, more preferably 25 to 40 and most preferably, from 30 to 40 repeating units in the backbone. In certain embodiments, the PEG side chains are each at least 3 kDa, preferably greater than 5 kDa. In other embodiments the PEG side chains range from 5 kDa to 40 kDa. In certain embodiments, the PEG brush polymer includes from 25 to 60 side chains attached to the polymer backbone and each side chain is at least 10 kDa. The overall molecular weight of the PEG brush polymer side chains is about 75 to 500 kDa. In a preferred embodiment, the pacRNA is designed with 30 PEG_{10 kDa} repeating units.

The PEG brush polymer can be made by any suitable method. For example, PEG brush polymers may be synthesized via sequential ring opening metathesis polymerization (ROMP) of norbornenyl bromide (N-Br) and norbornenyl PEG, for example, followed by azide substitution of the bromide. The resulting PEG brush polymer is a diblock structure, with the first block serving as a reactive region for nucleic acid conjugation, and the second longer block creating the brush architecture and the steric congestion needed to protect the nucleic acid.

The brush polymer structure can also be synthesized by atom transfer radical polymerization (ATPR) (See Neugebauer, D., Zhang, Y., Pakula, T., Sheiko, S. S., and Matyjaszewski, K. “Densely-grafted and double-grafted PEO brushes via ATRPA route to soft elastomers,” Macromolecules, 2003, 36(18), 6746-6755) or reversible addition—fragmentation chain-transfer polymerization (RAFT) (See Warren, N. J., and Armes, S. P. Polymerization-induced self-assembly of block copolymer nano-objects via RAFT aqueous dispersion polymerization. J. Am. Chem. Soc., 2014, 136(29), 10174-10185), for example.

Other methods for synthesizing various polymer brushes are known in the art. See for example, Hensarling et al., “Clicking” Polymer Brushes with Thiol-yne Chemistry: Indoors and Out, J. AM. CHEM. SOC. 2009, 131, 14673-14675; Prog. Polym. Sci. 25 (2000) 677-710; B. Zhao and W. J. Brittain, Polymer Brushes: surface immobilized macromolecules, Prog. Polym. Sci. 25 (2000) 677-710; US Patent Application No. 2018/0230467, each of which is incorporated herein in their entirety.

The PEG brush polymer generally has at least one RNA strand covalently bound to the brush polymer backbone. In certain embodiments, one to ten RNA strands, e.g., siRNA strands, are bound to the polymer backbone. A preferred pacRNA is designed with 30 PEG_{10 kDa} repeating units and less than five strands of siRNA, e.g., 1-4 RNA strands or 1-2 RNA strands.

In general, the RNA component of pacRNA is from about 3 to about 30 nucleotides in length. It is also contemplated that the RNA oligonucleotide is about 5 to about 75 nucleotides in length, about 5 to about 70 nucleotides in length, about 3 to about 65 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the RNA oligonucleotide is able to achieve the desired result. In a preferred embodiment, the RNA oligonucleotide is an siRNA of from 20 to 25 nucleotides in length.

The RNA component of the pacRNA can be single stranded or double stranded RNA. RNA oligonucleotides contemplated for attachment to a brush polymer backbone include those which modulate expression of a gene product expressed by a target polynucleotide. Accordingly, antisense oligonucleotides which hybridize to a target polynucleotide and inhibit translation, siRNA oligonucleotides which hybridize to a target polynucleotide and initiate an RNAse activity (for example RNAse H), triple helix forming oligonucleotides which hybridize to double-stranded polynucleotides and inhibit transcription, and ribozymes which hybridize to a target polynucleotide and inhibit translation, are contemplated.

Modification of the RNA is possible but not essential; native RNA (without further chemical modifications or formulation beyond PEGylation) can be used in the pacRNA complex because the pacRNA complex increases the enzymatic stability of the RNA. Thus, the pacRNA bypasses the need for phosphorothioates, which allows the pacRNA to avoid renal clearance.

Each pacRNA has the ability to hybridize with one or more target polynucleotides having a sufficiently complementary sequence to the RNA component of the pacRNA. For example, if a specific mRNA is targeted, a single brush polymer-RNA conjugate carrying a plurality of RNAs has the ability to hybridize with multiple copies of the same transcript. In one embodiment, methods are provided wherein the pacRNA is functionalized with identical RNA oligonucleotides, i.e., each oligonucleotide has the same length and the same sequence. In other embodiments, the pacRNA is functionalized with two or more oligonucleotides, which are not identical, i.e., at least one of the attached RNAs differs from at least one other attached RNA in that it has a different length and/or a different sequence or modification. In embodiments where different oligonucleotides are attached to the brush polymer, these different oligonucleotides hybridize with the same single target polynucleotide, but at different locations, or hybridize with different target polynucleotides which encode different gene products. Accordingly, in various aspects of the invention, a single functionalized pacRNA may be used to inhibit expression of more than one gene product. RNA oligonucleotides are thus used to target specific polynucleotides, whether at one or more specific regions in the target polynucleotide, or over the entire length of the target polynucleotide as the need may be to affect a desired level of inhibition of gene expression.

“Hybridize” and “hybridization” mean an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions. It will be understood by those of skill in the art that the degree of hybridization is less significant in the disclosed technology than a resulting degree of inhibition of gene product expression.

The RNA oligonucleotides are generally designed with knowledge of the target sequence or sequences. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991), incorporated herein by reference. Solid-phase synthesis methods are generally used to synthesize the RNA component of the pacRNA, while enzymatic synthesis is also contemplated.

The RNA oligonucleotide is bound to the brush polymer backbone in such a way that the oligonucleotide is released from the brush polymer after the pacRNA enters a cell. In general, the RNA oligonucleotide can be released from the brush polymer using either chemical methods, photon release (i.e., irradiating cells in which pacRNAs have entered using electromagnetic wavelengths selected on the basis of pacRNA size), changes in ionic or acid/base environment or a cleavable bond, such as an enzymatic or hydrolytically cleavable bond or a bioreductively cleavable linkage or use of an acid-labile or redox-labile linker, for example.

In one embodiment, the RNA oligonucleotide is attached to the brush polymer backbone via a redox-labile moiety and once the functionalized pacRNA is taken into the cell, the oligonucleotide is released from the polymer backbone. For example, a pacRNA structure can be synthesized to feature a redox-labile linker containing a disulfide bond, e.g., dibenzocyclooctyne-N-hydroxysuccinimidyl ester (DBCO-SS-HNS). Once the pacRNA enters the cells, the disulfide bond may be cleaved by glutathione inside the cells and free dsRNA (or ssRNA) is released to perform gene regulation functionality. This aspect is particularly useful in instances where the intent is to saturate the cell with for example, an siRNA and release from the brush polymer backbone would improve kinetics and resolve potential steric hindrance problems. Preparation and use of RNAi for modulating gene expression is well known in the art.

In various aspects of the invention, the target polynucleotide is eukaryotic, prokaryotic, or viral. In various embodiments, the target polynucleotide is an mRNA encoding a gene product and translation of the gene product is inhibited by the pacRNA, or the target polynucleic acid is DNA in a gene encoding a gene product and transcription of the gene product is inhibited. The target polynucleotide may be a DNA that encodes a gene product being inhibited or may be complementary to a coding region for a gene product. In still other embodiments, the target DNA encodes a regulatory element necessary for expression of a gene product. “Regulatory elements” include, but are not limited to enhancers, promoters, silencers, polyadenylation signals, regulatory protein binding elements, regulatory introns, ribosome entry sites, and the like. In still other embodiments the target polynucleotide is a sequence which is required for endogenous replication.

Target regions within a target DNA include any portion of the target nucleic acid, such as the 5′ untranslated region (5′UTR) of a gene, the portion of an mRNA in the 5′ direction from the translation initiation codon, including nucleotides between the 5′ cap site and the translation initiation codon of an mRNA (or corresponding nucleotides on the gene), and the 3′ untranslated region (3′UTR), the portion of an mRNA in the 3′ direction from the translation termination codon, including nucleotides between the translation termination codon and 3′ end of an mRNA (or corresponding nucleotides on the gene).

In some embodiments of the aspects of the invention, the target nucleic acid is a gene or RNA transcript specific to a cancer cell or which is over-expressed in cancer cells, particularly a gene that is over-expressed in several types of cancer cells. In other embodiments, the target nucleic acid is a gene that is unique to a cancer cell or particular disorder or disease state.

The pacRNAs described herein may be used to diagnose, prevent, treat or manage certain diseases or bodily conditions. In some cases, the pacRNA structures are both a therapeutic agent and a diagnostic agent. Therapeutic methods provided herein embrace those which result in essentially any degree of inhibition of expression of a target gene product.

In some embodiments, pacRNAs described herein may be used as intracellular diagnostic agents. The ability to deliver RNAs intact to the cell cytoplasm provides an opportunity to not only regulate RNA targets, but also to detect them. For instance, in some embodiments, delivery of a pacRNA having 3′ and/or 5′ detectable markers is used to detect the presence of target RNA.

The inventive pacRNAs may be used in “pharmaceutical compositions” or “pharmaceutically acceptable” compositions, which comprise a therapeutically effective amount of one or more of the pacRNAs described herein, formulated together with one or more pharmaceutically acceptable carriers, additives, and/or diluents. The pharmaceutical compositions described herein may be useful for diagnosing, preventing, treating or managing a disease or bodily condition such as cancer or bacterial or viral infection, for example. It should be understood that any pacRNA described herein can be used in such pharmaceutical compositions.

Pharmaceutical compositions containing the pacRNAs may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the pacRNA from one organ, or portion of the body, to another organ, or portion of the body. Each carrier is “acceptable” in the sense of being compatible with the other ingredients of the formulation and not injurious to the patient when administered in doses sufficient to provide a therapeutically effective amount of the brush polymer-oligonucleotide conjugate.

The pacRNA and compositions containing pacRNA may be orally administered, parenterally administered, subcutaneously administered, and/or intravenously administered for carrying out the methods of the invention. In certain embodiments, a pacRNA or pharmaceutical composition containing a pacRNA is administered orally. In other embodiments, the pacRNA or pharmaceutical composition containing a pacRNA is administered intravenously or via injection into a target site such as a tumor or muscle. Alternative routes of administration include sublingual, intramuscular, and transdermal administrations.

In some embodiments of the invention, the pacRNA or composition containing pacRNA is applied to a biological sample, such as a blood sample or other tissue obtained from a subject, such as a human or other mammal. In this case, the pacRNA is optionally labelled with one or more detectable labels.

In another aspect of the invention, there is provided a method for inhibiting expression of a gene product encoded by a target polynucleotide comprising contacting the target polynucleotide with a brush polymer-RNA conjugate of the invention. In various embodiments, expression of the gene product is inhibited in vivo or expression of the gene product may be inhibited in vitro. In other embodiments of this aspect, the brush polymer-RNA conjugate comprises a PEG brush polymer having a one or more of a double stranded RNA molecule (siRNA) attached to the polymer backbone, preferably by a releasable linker such as a redox-labile linker containing a disulfide bond or an acid-labile linker. In this manner, the pacRNAs of the invention may be used to treat disease, such as cancer.

In another aspect, methods are provided for promoting cellular uptake of an RNA oligonucleotide in a subject or biological sample, comprising delivering a pacRNA structure to the subject or the biological sample in an effective amount for promoting cellular uptake of the RNA. The pacRNA may comprise one or a plurality of RNA oligonucleotides linked to the backbone of a PEG brush polymer, preferably via a releasable linker as described herein above. The RNA oligonucleotide may be single or double stranded. Preferably, the RNA oligonucleotide(s) is complementary to a target nucleotide sequence associated with a disease state or disorder.

Also provided are kits for inhibiting gene expression of a target polynucleotide. In one embodiment of this aspect, the kit contains at least one type of pacRNA as described herein or a plurality of types of pacRNAs providing a plurality of different oligonucleotides as described herein attached to a brush polymer backbone. The oligonucleotides on the first type of pacRNA have one or more sequences complementary (or sufficiently complementary as disclosed herein) to one or more sequences of a first portion of a target polynucleotide. The kit optionally includes one or more additional types of pacRNA which have a sequence complementary to one or more sequences of a second portion of the target polynucleotide or to a second (or third, etc.) target sequence. In some embodiments of the kits provided, oligonucleotides include a detectable label or the kit includes a detectable label which can be attached to the RNA oligonucleotides or the brush polymer.

### EXAMPLES

### Example 1

### Synthesis and Analysis

Materials and methods: ω-Amine polyethylene glycol (PEG) methyl ether (Mₙ=10 kDa, PDI=1.05) was purchased from JenKem Technology USA. Dibenzocyclooctyne-S—S—N-hydroxysuccinimidyl ester (DBCO-S—S—NHS) was purchased from Sigma-Aldrich Co. Phorsphoramidites and supplies for RNA synthesis were obtained from Glen Research Co. Dulbecco's Modified Eagle Medium (DMEM) and 3-(4,5-dimethyl-thiazol-2-yl)-2,5-diphenyl tetrazolium bromide (MTT) were obtained from Sigma-Aldrich CO. Roswell Park Memorial Institute (RPMI) 1640 medium was obtained from Corning. Human SKBR3 and SKOV3 cancer cell lines were purchased from American Type Culture Collection (Rockville, Md., USA). All other materials were obtained from Fisher Scientific Inc., Sigma-Aldrich Co., or VWR International LLC. and used as received unless otherwise indicated.

Instrumentation: ¹H and ¹³C NMR spectra were recorded on a Varian 400 MHz NMR spectrometer (Varian Inc., CA, USA). MALDI-TOF MS measurements were performed on a Bruker Microflex LT mass spectrometer (Bruker Daltonics Inc., MA, USA). Fluorescence spectroscopy was carried out on a Cary Eclipse fluorescence spectrophotometer (Varian Inc., CA, USA). Fourier transform infrared (FTIR) spectra were recorded on a Bruker Tensor FT-IR spectrometer (Bruker Corporation, MA, USA) by KBr sample holder method. Reverse-phase HPLC was performed using a Waters Breeze 2 HPLC system coupled to a Symmetry® C18 3.5 μm, 4.6×75 mm reverse phase column and a 2998 PDA detector, using TEAA buffer (0.1 M) and HPLC-grade acetonitrile as mobile phases. N,N-Dimethylformamide (DMF) GPC was performed on a TOSOH EcoSEC HLC-8320 GPC system (Tokyo, Japan) equipped with a TSKGel GMH_{HR}-H, 7.8×300 mm column and RI/UV-Vis detectors. HPLC-grade DMF with 0.04 M LiBr was used as the mobile phase, and samples were run at a flow rate of 0.5 mL/min. GPC calibration was based on polystyrene standards (706 kDa, 96.4 kDa, 5970 Da, 500 Da). Aqueous GPC measurements were carried out on a Waters Breeze 2 GPC system equipped with an Ultrahydrogel™ 500, 7.8×300 mm column and a 2998 PDA detector (Waters Co., MA, USA). Sodium nitrate solution (0.1 M) was used as the eluent running at a flow rate of 0.8 mL/min. Gel electrophoresis was performed using 0.5% agarose gel in 0.5×tris/borate/EDTA (TBE) buffer with a running voltage of 100 V. Gel images were acquired on an Alpha Innotech Fluorochem Q imager. DLS and zeta potential data were recorded on a Malvern Zetasizer Nano-ZSP (Malvern, UK). TEM samples were measured on a JEOL JEM 1010 electron microscope utilizing an accelerating voltage of 80 kV.

Synthesis of Monomers

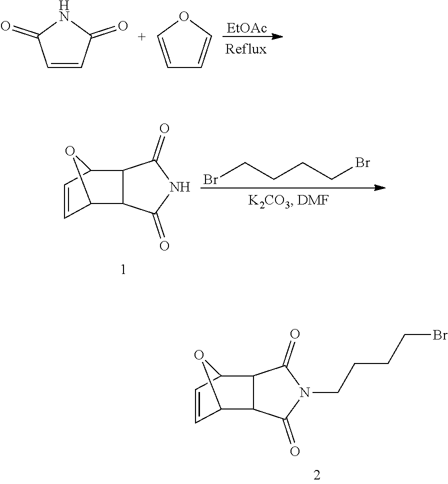

Compound 1. In a round bottom flask, 2.0 g (20.6 mmol) of maleimide and 1.54 g (22.6 mmol) of furan were dissolved in 20 mL ethyl acetate. The solution was refluxed for 4 h, and a white solid, 1, precipitated from the reaction mixture during the course of the reaction. The solids were isolated by filtration, washed with ethyl ether, and dried under vacuum, to provide 7-oxabicyclo(2.2.1)hept-5-ene-2,3-dicarboxamide (1).

¹H-NMR (400 MHz, CDCl₃): δ 8.14 (s, 1H, —CNHC—), 6.52 (s, 2H, CH═CH), 5.31 (s, 2H, CHOCH), 2.99 (s, 2H, CH—CH); ¹³C-NMR (400 MHz, CDCl₃): δ 176.2, 136.8, 81.2, 48.9.

Compound 2. 2.59 g (12 mmol) of 1,4-dibromobutane, 2.07 g of K₂CO₃ (15 mmol), and 5 mL DMF were added to a round bottom flask, to which a solution of 1 (0.5 g, 3 mmol) in 5 mL DMF was added dropwise over a period of 30 min with stirring. The reaction mixture was allowed to stir overnight at room temperature. Silica gel chromatography (3:1 v:v hexane:EtOAc) was used to purify 2. Upon drying under vacuum, 2 appears as a white solid.

¹H-NMR (400 MHz, CDCl₃): δ 6.51 (s, 2H, CH═CH), 5.26 (s, 2H, CHOCH), 3.51 (t, 2H, NCH₂), 3.41 (t, 2H, CH₂Br), 2.84 (s, 2H, CH—CH), 1.84-1.71 (m, 4H, CH₂—CH₂); 13C-NMR (400 MHz, CDCl₃): δ 176.5, 136.8, 81.2, 47.6, 38.1, 33.1, 29.8, 26.4.

Synthesis of norbornenyl PEG (4). Norbornenyl PEG (4) was synthesized in two steps as described previously (D. Castanotto, J. J. Rossi, The promises and pitfalls of RNA-interference-based therapeutics, Nature 457, 426-433 (2009).). First, the norbornenyl NHS ester was prepared by esterification of norbornenyl carboxylic acid with N-hydroxysuccinimide (NHS) using the activating reagent, ethyl(dimethylaminopropyl) carbodiimide (EDC). Then, ω-amine-terminated poly(ethylene glycol) methyl ether (mPEG-NH₂, 1.0 g, 0.1 mmol, Mₙ=10 kDa) and norbornenyl NHS ester (30.5 mg, 0.13 mmol) were stirred in a dichloromethane solution (20 mL) containing of N,N-diisopropyl ethyl amine (DIPEA, 16 mg, 0.1235 mmol) for 6 h at room temperature. This reaction was monitored by MALDI-TOF MS until all mPEG-NH₂ was consumed. The solution was concentrated and precipitated into cold diethyl ether three times, and the final product was dried under vacuum for 48 h.

Synthesis of diblock brush copolymer (6). A modified 2nd generation Grubbs' catalyst was synthesized according to a previously reported method (de Fougerolles, H.-P. Vornlocher, J. Maraganore, J. Lieberman, Interfering with disease: a progress report on siRNA-based therapeutics, Nature Rev. Drug Discov. 6, 443-92 453 (2007)). A solution of norbornenyl bromide (2, 5 equiv.) in deoxygenated dichloromethane was added by gastight syringe into a Schlenk flask under N₂. The solution was cooled to −20° C. in an ice-salt bath, to which modified Grubbs' catalyst (1 equiv.) in deoxygenated dichloromethane was added via a gastight syringe. The reaction mixture was stirred vigorously for 30 min. Thin-layer chromatography (TLC) confirmed the complete consumption of the monomer. Then, a solution of 4 (30 equiv.) in deoxygenated dichloromethane was added by gas-tight syringe to the reaction. The reaction mixture was further stirred for 6 h, before addition of several drops of ethyl vinyl ether (EVE) to remove the chain-end catalyst. The mixture was stirred overnight and then precipitated into cold diethyl ether three times. The resulting white solids were dried under high vacuum. Next, the polymer (5) was treated with an excess of sodium azide in DMF overnight at room temperature. The resulting solution was dialyzed against Nanopure™ water for 24 h, lyophilized, re-dissolved in Nanopure™ water, and injected into an aqueous GPC for collection of the fractions containing the brush polymer. The final polymer (6) was further desalted using a NAP-10 column (G.E. Healthcare, IL, USA). FT-IR confirmed the successful incorporation of azide functionalities. To quantify the number of azide groups per copolymer available for coupling, 5 nmol of the polymer was dissolved in Nanopure™ water (200 μl) and conjugated with alkyne-modified fluorescein (Lumiprobe, 0.207 mg, 500 nmol) using copper catalyst (CuSO₄.5H₂O, 500 nmol; tris(3-hydroxypropyltriazolylmethyl)amine, THPTA, 600 nmol; sodium ascorbate, 2.5 μmol. The reaction mixture was gently shaken on an Eppendorf Thermomixer at room temperature overnight. Thereafter, the solution was dialyzed against a NaCl solution (0.15 M) using dialysis tubing with a MWCO of 6-8 kDa for 48 h. The UV-Vis absorption of the polymer solution at 491 nm was measured and compared with a standard curve. The number of fluorescein molecules per polymer was calculated based on the known polymer concentration. Approximately 5 fluorescein tags were attached to each diblock brush copolymer.

Quantitation of the Azide Groups in Brush Polymers

In a 1.5 mL microcentrifuge tube, brush polymer (5 nmol) was dissolved in 200 μL Nanopure™ water, to which fluorescein-alkyne (Lumiprobe, 0.207 mg, 500 nmol), copper(II) sulfate pentahydrate (CuSO₄.5H₂O, 500 nmol, 5 μL 100 mM aqueous solution), Tris(3-hydroxypropyltriazolylmethyl)amine (THPTA, 600 nmol, 6 μL 100 mM aqueous solution), and sodium ascorbate (2.5 μmol) were added. The reaction mixture was shaken on an Eppendorf shaker at room temperature overnight, before being dialyzed against a NaCl solution (0.15 M) using dialysis tubing with a MWCO of 6-8 kDa for 48 h. The UV-Vis absorption of the polymer solution at 491 nm was measured and compared with a standard curve. The number of fluorescein molecules per polymer was calculated based on the known polymer concentration.

Synthesis of Cy5.5 Labeled Brush Polymer

A sample of azide-functionalized diblock PEG brush polymer (10 mg) was dissolved in DMF (1 mL) and placed in a glass bottle. Cy5.5-alkene (0.5 mg), copper(I) and tris-(benzyltriazolylmethyl)amine (TBTA) were then added. The reaction mixture was kept in the dark for 24 h and subsequently dialyzed against ^{˜}6 exchanges of water (2 L) over the ensuing 3 days, using a dialysis bag with a molecular cutoff of 3500 Da. The reaction product was subsequently frozen and lyophilized prior to further use.

Oligonucleotide Synthesis

RNA was synthesized on a Model 391 DNA synthesizer (Applied Biosystems, Inc., Foster City, Calif.) using standard solid-phase phosphoramidite methodology. All RNA strands were cleaved from the CPG support using ammonium hydroxide/40% aqueous methylamine (1:1) solution at 65° C. for 10 minutes. The 2′-O-triisopropylsilyloxymethyl (TOM) protecting group was removed by treatment with triethylamine trihydroflouride (TEA.3HF) in dimethylsulfoxide (DMSO) at 65° C. for 2.5 h. Finally, both DNA and RNA were purified by reverse-phase HPLC liquid chromatography. The successful syntheses of all RNA sequences were verified by MALDI-TOF MS.

Synthesis and Purification of DBCO-SS-RNA

Amine-modified RNA strands were synthesized using standard solid-phase phosphoramidite chemistry. Next, the lyophilized amino-modified RNA was dissolved in 100 μl of NaHCO₃ (0.1 M) buffer, to which 0.5 mg DBCO-SS-NHS was added via 100 μl DMSO solution. The reaction mixture was shaken at 0° C. overnight. The products (DBCO-SS-RNA or DBCO-RNA) were purified by reverse-phase HPLC liquid chromatography.

Synthesis of pacRNA

In a typical procedure, azide-functionalized brush copolymers were dissolved in 200 μL Nanopure™ water and then added to the DBCO-modified RNA aqueous solution (100 μL). The mixture was shaken gently for 24 h at 40° C. on an Eppendorf Thermomixer. After that, the product was purified by aqueous GPC. After purification, the conjugate was desalted by a NAP-10 desalting column (G.E. Healthcare) and was lyophilized to yield a white powder (or green/red powders for fluorescein and Cy3-labeled conjugates). To form double-stranded (ds) oligonucleotides, the solid powders were dissolved in phosphate-buffered saline (PBS), followed by the addition of 1 equiv. of the single-stranded (ss) complementary strand. The solutions were annealed by heating to 80° C. and cooled to room temperature in a thermally insulated container over 24 hours.

Synthesis of Off-On Fluorescent Reporter pacRNA for Intracellular Release Monitoring.

Alkyne-modified dabcyl (quencher) was first synthesized by coupling dabcyl NHS ester and propargylamine via amidation chemistry. The quencher was then reacted with the brush copolymer (6) via copper-catalyzed click chemistry (vide supra, 5:1 mol:mol alkyne:N3). The reaction mixture was stirred overnight, dialyzed against Nanopure™, and was further purified by aqueous GPC. The fractions containing the conjugate were collected and desalted by dialysis. The final solution was lyophilized to yield an orange powder. UV-Vis spectroscopy indicated that there was ^{˜}4 dabcyl molecules per brush polymer. Next, the remaining N3 groups on the dabcyl-containing brush polymer were reacted with an excess (^{˜}2 equiv. to N3) of DBCO-modified RNA (fluorescein-labeled) by gentle shaking on an Eppendorf Thermomixer at 45 QC for 24 hours. The final product was purified by aqueous GPC, desalted by a NAP-10 column, and lyophilized. UV-vis quantification indicates ^{˜}1 RNA strand per brush polymer.

### Example 2

### Analysis

Molecular dynamics (MD) simulation. MARTINI coarse-grained (CG) force-field with explicit water was used for MD simulation of pacRNA . The force field incorporates four heavy atoms with similar chemical identities into one CG bead, and therefore reduces the freedoms of the molecules needed to calculate. Bonded parameters are defined based upon molecular structure, while non-bonded parameters, including van der Waals and electrostatic forces, are derived from free energy partitioning between polar and organic solvents. The MARTINI version of PEG was developed by Lee et al (H. Lee, A. H. de Vries, S.-J. Marrink, R. W. Pastor, A coarse-grained model for polyethylene oxide and polyethylene glycol: conformation and hydrodynamics, J. Phys. 13 Chem. B 113, 13186-94 (2009). The atomistic to CG mapping is 3:1 for the PEG monomer. This mapping ratio deviates from the standard MARTNI mapping scheme due to the size of PEG monomer. Herein, the PEG monomer is represented by an SN0 particle in the CG force field. The LI interaction parameters between PEG and water are σ=0.47 nm and ε=4.0 kJ/mol. The MARTINI version of RNA has been recently developed based upon its DNA predecessor (49). The tertiary structure of RNA was constrained using an elastic network. Time step of CG MD simulations was set to be 0.010 ps with periodic boundary conditions. The system was controlled using an NPT ensemble. The temperature was maintained at 310 K using Berendsen temperature coupling while the pressure was controlled at 1 atm by the Berendsen pressure rescaling method (50). Cutoff distance of van der Waals and short-ranged electrostatic interactions was set at 1.2 nm. Long-ranged electrostatic interactions were not considered. All simulations were performed on high-performing computing clusters using the GROMACS 5.0.5 package.

Hybridization Kinetics Assay

Free RNA, pacRNA, pacRNA_{NClv} and pacRNA_{Clv} and Y-shaped PEG (40 kDa)-RNA conjugate (all fluorescein-labeled) were dissolved in PBS buffer (pH=7.4) at a final RNA concentration of 100 nM, respectively. A total of 1 mL solution for each sample was transferred to a fluorescence cuvette. Then a complementary dabcyl-RNA strand or a non-complementary dummy strand (2 equiv.) was added via 1 μL of PBS solution into the fluorescence cuvette. The fluorescence of the mixed solution (ex=490 nm, em=520 nm) was monitored every 3 seconds before and after mixing for 60 min. The endpoint is determined by adding a large excess of complementary dabcyl-RNA to the mixture followed by incubation for more than 2 h. The kinetics plots were normalized to the endpoint determined for each sample.

Nuclease Degradation Kinetics Assay

Free RNA, pacRNA_{NClv}, and pacRNA_{Clv}, and Y-shaped PEG (40 kDa)-RNA conjugate (1 μM, all fluorescein-labeled) were mixed with their complementary dabcyl-labeled RNA (2 μM) in PBS buffer, respectively. The mixed solutions were gently shaken at room temperature overnight. Subsequently, 100 μL of each sample was withdrawn and diluted to 100 nM with assay buffer.

Then RNase III was added to the solution at a final concentration of 0.4 unit/mL, and the fluorescence of each sample was monitored every 3 seconds (ex=490 nm, em=520 nm) for 5 h. The endpoint of each sample was determined by adding a large excess of RNase III (ca. 2 units/mL) to the mixture, and the fluorescence was monitored until no additional increase was observed. The kinetics plots were normalized to the endpoints, and all measurements were repeated at least three times.

In Vitro siRNA Release Measurements

PacRNA_{Clv}, samples (20 μL, 100 nM) were treated with 10 mM DTT in 1×PBS at 37° C. for 5 min, 30 min, 60 min, 120 min and 240 min, respectively. Thereafter, RNA release was monitored by gel electrophoresis, which was carried out using 0.5% agarose gel in 0.5× TBE buffer with a running voltage of 120 V. pacRNA_{NClv} was treated with 10 mM DTT for 240 min under the identical condition, which was set as a control. The amount of RNA released was determined by using the software ImageJ. All RNA release experiments were conducted in triplicate and the results are expressed as the average data with standard deviations.

Cell culture: SKOV3 (a human ovarian cancer cell line) and HDF (Human Dermal Fibroblasts) cells were cultured in DM EM supplied with 10% fetal bovine serum, 1% L-glutamine and 1% antibiotics at 37° C. in a humidified atmosphere containing 5% CO₂. Human SKBR3 cells (breast adenocarcinoma cell line) were cultured in complete RPMI medium (RPMI 1640 containing 10% FBS, 1% L-glutamine and 1% antibiotics) at 37° C. in 5% CO₂.

Cell uptake: The cell uptake ability of pac-RNA was investigated on flow cytometry and confocal laser scanning microscopy (CLSM) using SKOV3 and SKBR3 cells. For flow cytometry, cells were seeded in 6-well plates at 2.0×10⁵ cells per well in 2 mL complete DMEM and cultured for 24 h at 37° C. with 5% CO₂. Then, Cy3-labeled PO ds RNA, pacRNA_{Clv}, pacRNA_{NClv}, PS ss RNA, and PS ds RNA (100 nM-2 μM equiv. of siRNA) dissolved in DM EM or RPMI culture medium was added, and cells were further incubated at 37° C. for 4 h. Subsequently, cells were washed with PBS 3× and suspended by treatment with trypsin. Thereafter, 2 mL of PBS was added to each culture well, and the solutions were centrifugated for 5 min (1000 rpm). Cells were then resuspended in 0.5 mL of PBS for flow cytometry analysis on a BD FACS Calibur flow cytometer. Data for 1.0×104 gated events were collected. The experiments were carried out in triplicate.

Flow Cytometry: SKOV3 or SKBR3 cells were seeded in 6-well plates at 2.0×10⁵ cells per well in 1 mL complete DMEM and cultured for 24 h at 37° C. and 5% CO₂. Then Cy3-labeled free RNA, pacRNA_{NClv}, and pacRNA_{Clv} (1 μM equivalent of RNA) dissolved in DMEM or RPMI culture medium were added into different wells and the cells were further incubated at 37° C. for 4 h. Subsequently, culture medium was removed, and cells were washed with PBS twice and treated with trypsin. Thereafter, 2 mL of PBS was added to each culture well, and the solutions were centrifugated for 5 min (1000 rpm). After the supernatants were removed, the cells were resuspended in 0.5 mL of PBS. Data for 1.0×10⁴ gated events were collected and analysis was performed on BD FACS Calibur flow cytometer.

Confocal Laser Scanning Microscopy (CLSM): SKOV3 or SKBR3 cells were seeded in 24-well glass bottom plates at 1.0×10⁵ cells per well and cultured for 24 h, followed by removing culture medium and adding Cy3-labeled free RNA, pacRNA_{NClv}, or pacRNA_{Clv} at a final RNA concentration of 1 μM. The cells were incubated at 37° C. for 4 h. The culture medium was removed and cells were washed with PBS for three times. Subsequently, the cells were fixed with 4% paraformaldehyde for 30 min at room temperature, and then rinsed with PBS for three times. Finally, the cells were stained with Hoechst 33342 for 10 min and rinsed with PBS for three times. The cells were imaged on an LSM-700 confocal laser scanning microscope (Carl Zeiss Ltd., Cambridge, UK). Imaging settings were identical for free RNA- and pacRNA-treated cells.

Intracellular siRNA release: Intracellular siRNA release was analyzed using CLSM. SKOV3 or SKBR3 cells were seeded in 24-well plates at 1.0×10⁵ cells per well and incubated at 37° C. for 12 h. Dabcyl- and fluorescein-labeled pacRNA_{NClv}, or pacRNA_{Clv} were then added to these plates at a final RNA concentration of 1 μM. After that, the plates were then placed in either a 37° C. incubator for 4 h. For each plate, the media was then removed and the cells were washed twice with cold PBS, stained with Hoechst 33342 for 10 min, and rinsed with PBS for three times. Fluorescein was then measured using an LSM-700 confocal laser scanning microscope (Carl Zeiss Ltd., Cambridge, UK). Imaging settings were identical for pacRNA_{NClv}, and pacRNA_{Clv}, -treated cells.

MTT assay: The cytotoxicity of pacRNA_{NClv} and pacRNA_{Clv} was evaluated with the MTT assay against SKOV3 and SKBR3 cells. Briefly, SKOV3 or SKBR3 cells were seeded into 96-well plates at 1.0×10⁴ cells per well in 200 μL DMEM and cultured for 24 h. The cells were then treated with free RNA, pacRNA_{NClv} or pacRNA_{Clv} at varying concentrations of total RNA (50 nM, 100 nM, 250 nM, 500 nM and 1000 nM). Cells without any treatment were used as a negative control. Lipofectamine 2000 (Invitrogen) was used as a positive control following the instruction of the manufacturer. After incubating 48 h, 20 μL of 5 mg/mL MTT assays stock solution in phosphate buffered saline (PBS) was added to each well. The cells were incubated for 4 h and the medium containing unreacted MTT was removed carefully. The obtained blue formazan crystals were dissolved in 200 μL per well DMSO and the absorbance was measured in a BioTek® Synergy HT at a wavelength of 490 nm.

Hemolysis. A hemoglobin-free RBC (2% w/v) suspension was prepared by repeated centrifugation (2000 rpm for 10 min at 4° C.) and resuspension in ice cold PBS for a total of 3×. After the final resuspension, the concentration of RBCs was adjusted to 2% w/v. Thereafter, samples (free RNA, pacRNA_{Clv}, pacRNA_{NClv}, and Lipofectamine-complexed RNA, 1 μM equiv. RNA) were dissolved in PBS, added to the RBC suspension in 1:1 (v:v) ratio, and incubated for 1 h at 37° C. Complete hemolysis was attained using 2% v/v Triton-X, yielding the 100% control value. After incubation, centrifugation (2000 rpm for 10 min at 4° C.) was used to isolate intact RBCs, and the supernatants containing released hemoglobin were transferred to quartz cuvettes for spectrophotometric analysis at 545 nm. Results were expressed as the amount of hemoglobin released as a percentage of total.

Activated partial thromboplastin time. The aPTT assay was performed on a model BFT-2 coagulometer (Siemens, USA) to determine the clotting times for each sample. First, normal human plasma (50 μL) was incubated with aPTT-XL (50 μL, ThermoFisher, MA, USA) at 37° C. for 5 min. Thereafter, controls/samples (up to 60 μM of RNA) were added and the mixtures were further incubated for another 5 min. Finally, CaCl₂ (50 μL of 0.025 M) was added to each mixture to initiate the coagulation. The time until clot formation after the addition of CaCl₂ was automatically recorded by the coagulometer. All experiments were performed in triplicate.

Histochemical analyses. Following the anti-tumor study, mice were euthanized with CO₂, and tumors and major organs (heart, lung, liver, spleen and kidney) from each group were excised, fixed in 4% paraformaldehyde/PBS for 6 h, and placed into a 30% sucrose/PBS solution overnight at 4° C. The fixed tissues were paraffin-embedded and cut into 8 μm-thick sections with a cryostat. The sections were then processed with H&E staining. Immunohistochemistry targeting Bcl-2 was also carried out using mouse anti-Bcl-2 primary antibody (1:100 dilution, Invitrogen Co., CA, USA) and goat anti-mouse secondary antibody (1:1000 dilution, ThermoFisher, MA, USA). In addition, TUNEL assay was performed using the In Situ Cell Death Detection Kit (Roche, Switzerland) on sectioned tissues.

Western blotting: SKOV3 or SKBR3 cells were seeded in 12-well plates at a density of 2.0×10⁵ cells per well in 1 mL of DM EM complete medium and cultured for 24 h. The cells were treated with free RNA, pacRNA_{NClv}, pacRNA_{Clv}, scrambled pacRNA_{Clv}, and Lipofectamine-complexed RNA dissolved in DM EM at the same RNA concentration (1 μM) for 24 h. Thereafter, the culture medium was replaced with fresh, full growth medium and cells were further cultured for 48 h. The cells were harvested and whole cell lysates were collected in 100 μL of RIPA Cell Lysis Buffer with 1 mM phenylmethanesulfonylfluoride (PMSF, Cell Signaling Technology) following the manufacturer's protocol. Protein content in the extracts was quantified using a bicinchoninic acid (BCA) protein assay kit (Pierce, Germany). Equal amounts of proteins (30 μg/lane) were separated on 4-20% SDS-PAGE and electro-transferred to nitrocellulose membrane. The membranes were then blocked with 3% BSA (bovine serum albumin) in TBST (Tris buffered saline supplemented with 0.05% Tween-20) and further incubated with β-actin (1:1000 dilution) and Bcl-2 (1:1000 dilution) primary and secondary antibodies (5000:1 dilution, Invitrogen). Protein bands were detected by chemiluminescence using the ECL Western blotting substrate (Themo Scientific, USA) following the manufacturer's protocol.

Cell Apoptosis

SKOV3 cells were treated with samples/controls in the same fashion as those used in the western blotting study. For apoptosis analysis, both floating and attached cells were harvested, rinsed 3× with cold PBS, stained with Alexa Fluor®488 annexin V and propidium iodide (PI), and analyzed by flow cytometry to identify apoptotic cells.

In Vivo Biodistribution and Tumor Targeting Capability

All animal experiments were performed in accordance with the principles of care and use of laboratory animals and approved by the Animal Ethics Committee of Northeastern University. Non-invasive optical imaging systems were used to observe the real-time distribution and tumor accumulation ability of Cy5.5-labelled brush polymer or Cy5-labelled pacRNA_{NClv} and pacRNA_{Clv} at the RNA component. For in vivo imaging experiments, SKOV3 cells were induced in female Balb/c nude mice by subcutaneous injection of 2.0×10⁶ cells suspended in PBS. When the tumor volume reached approximately 200-300 mm³, the mice were administered with Cy5-labeleld RNA, Cy5.5-labelled brush polymer or Cy5-labelled pacRNA_{NClv} and pacRNA_{Clv} via tail vein injection, and scanned at 1, 4, 8 and 24 h using a fluorescence imaging system. After 24 h, mice were killed, tumors and other organs were removed and the biodistributions of these formulations were analyzed using a fluorescence imaging system.

Pharmacokinetic Studies

C57BL/6 mice were chosen to examine the pharmacokinetics of free RNA, pacRNA_{NClv}, pacRNA_{Clv} and the free brush polymer lacking an RNA component. Mice were randomly divided into four groups (n=4). Cy5-labeleld RNA, Cy5.5-labelled brush polymer and Cy5-labelled pacRNA_{NClv} or pacRNA_{Clv} solutions were intravenously administrated through the tail vein at a dose of 500 nmol/kg (RNA equivalent doses and/or brush polymer equivalent does), respectively. The blood samples (50 μL) were collected from the submandibular vein at 30 min, 2 h, 4 h, 10 h, and 24 h. The plasma was obtained by centrifugation at 3000 rpm for 15 min and stored at −20° C. The fluorescence intensity of the sample was measured in a BioTek® Synergy HT. The amounts of RNA and brush polymer were determined from standard curves previously obtained by analysis of blood samples containing known amounts of RNA and brush polymer.

In vivo antitumor efficacy: Tumors were produced in Balb/c female nude mice as described above. Mice were inspected for tumor appearance by observation and palpation. Tumor-bearing mice were randomly divided into 3 groups (each 4 mice): 1) the PBS group; 2) pacRNA_{NClv}; 3) pacRNA_{Clv}. Each sample was injected via the lateral tail vein once every 4 days for 28 days. The volume of tumors and weight of mice were measured before every treatment and the fourth day after the last administration to mice. Antitumor activity was evaluated in terms of tumor size (V=1/2ab²; a, long diameter; b, short diameter) by measuring two orthogonal diameters at various time points. Animals were sacrificed by cervical dislocation. Tumors were dissected and fixed with formalin for pathological section.

In vivo immune response: Innate immune responses following the injection of samples were evaluated using C57BL/6 mice. Samples and a positive control (lipopolysaccharide, 15 or 150 μg per mouse; n=4) were intravenously administrated through via the tail vein at equal RNA concentration (500 nmol/kg; free brush concentration equals that of the pacRNA). Eight hours post administration, blood samples were collected for cytokine analysis using ELISA kits (R&D Systems, Inc., MN, USA).

Statistics

All experiments were repeated at least three times. Data are presented as means±standard deviation. Statistical significance (p<0.05) was evaluated by using Student's t-test when only two groups were compared. If more than two groups were compared, evaluation of significance was performed using one-way analysis of variance (ANOVA) followed by Bonferroni's post hoc test. In all tests, statistical significance was set at p<0.05.

### Example 2

### pacRNA Synthesis

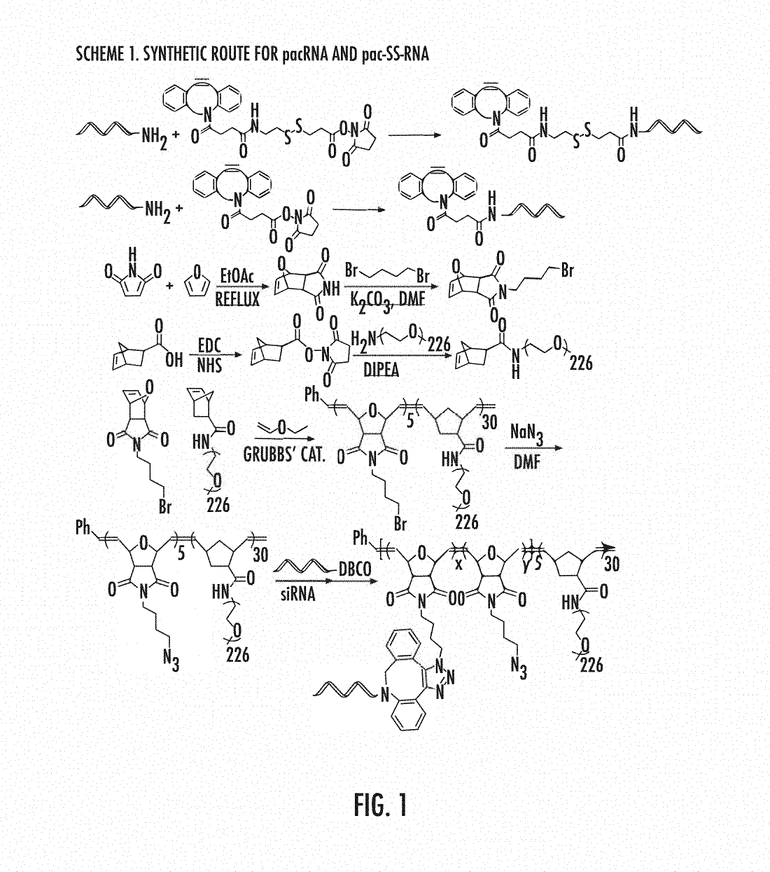

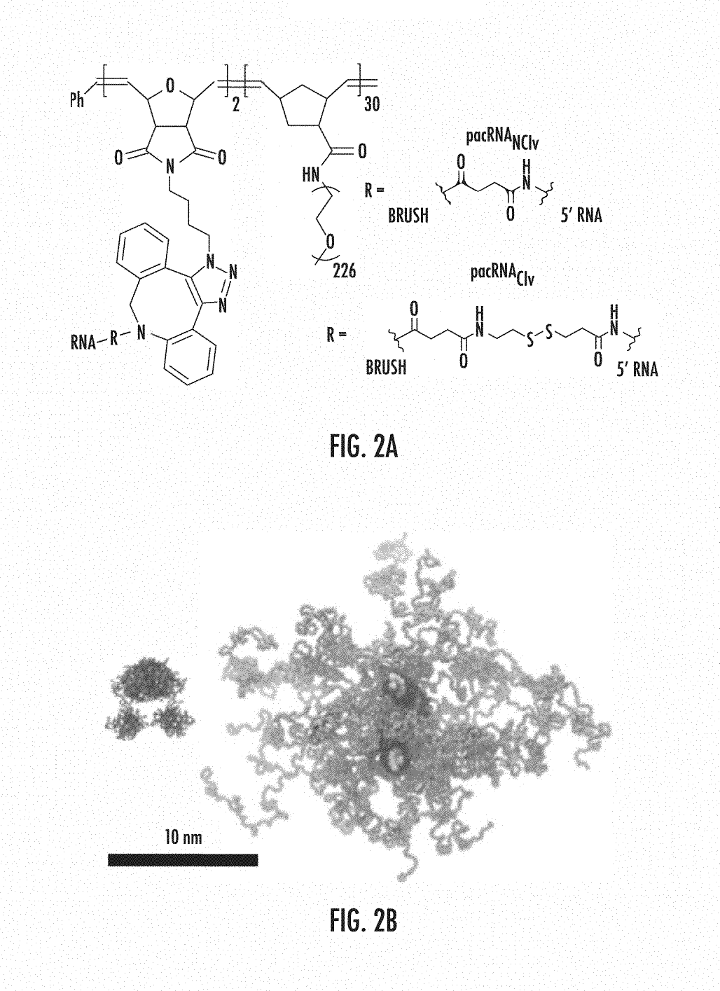

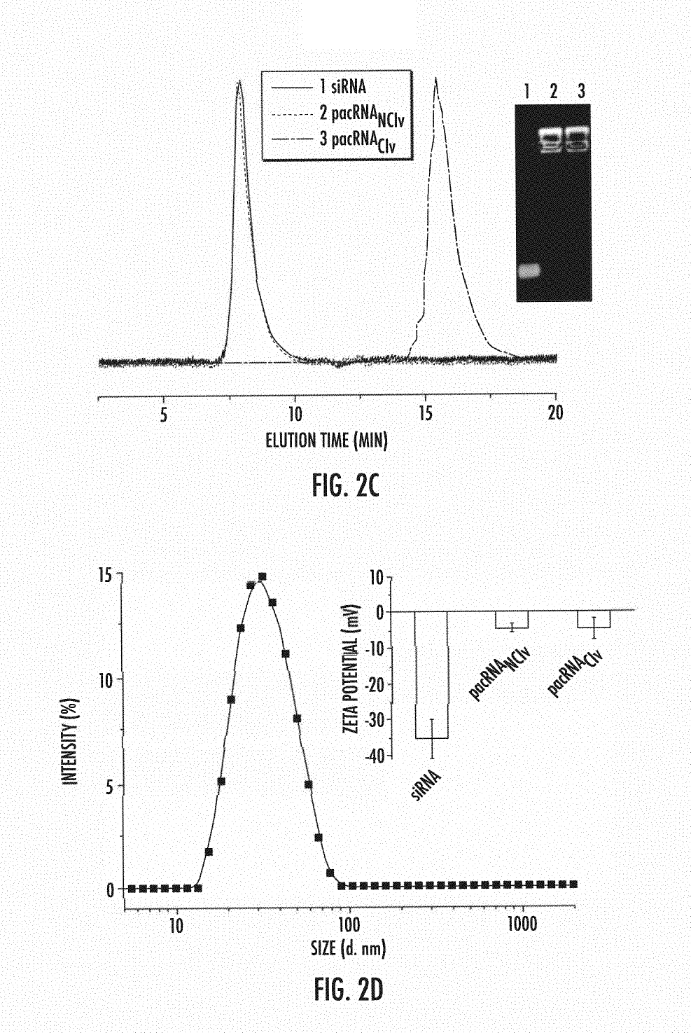

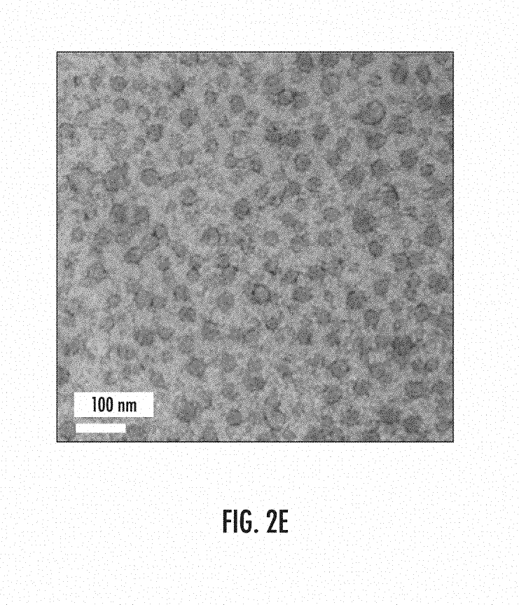

A pacRNA with 30 PEG_{10 kDa} repeating units and ^{˜}2 strands of siRNA was designed (FIGS. 2a-b). The brush polymer was synthesized via sequential ring-opening metathesis polymerization (ROMP) of 7-oxanorbornenyl bromide (ONBr) and norbornenyl PEG (NPEG), to yield a diblock architecture (pONBr₅-b-pNPEG₃₀), followed by azide substitution of the bromide (FIG. 1). The first, oligomeric block serves as a reactive region for RNA conjugation while the second, longer PEG block creates the steric congestion needed to shield the RNA. The guide strand of the siRNA was synthesized with a 5′ amine group, which was used to react with a cleavable linker (dibenzocyclooctyne-disulfide-N-hydroxysuccinimidyl ester) or a non-cleavable linker (dibenzocyclooctyne-N-hydroxysuccinimidyl ester). The resulting products were purified by reverse-phase HPLC and their structures were confirmed by MALDI-TOF MS (FIG. 1). Subsequently, the dibenzocyclooctyne-terminated guide RNA strands were coupled to the brush polymer via copper-free click chemistry, followed by hybridization with the passenger strand, to yield both the bioreductively cleavable conjugate (pacRNA_{Clv}) and the non-cleavable 09 version (pacRNA_{NClv}). The molecular weight of the final conjugate is 330 kDa, with a polydispersity index (PDI) of 1.2. The successful synthesis of the pacRNA is corroborated by aqueous gel permeation chromatography (GPC) and agarose gel electrophoresis (FIG. 2c). Note that the upward gel migration of the pacRNA is a consequence of the transient interaction of PEG with passing cations during electrophoresis, and not because of a net positive charge. Indeed, potential measurements indicate that the pacRNAs have a slight negative charge (−4.5 mV) in Nanopure [symbol] water, which is significantly below that of free siRNA (^{˜}35 mV, FIG. 2d inset). The pacRNAs exhibit a spherical morphology, with a dry-state diameter of ^{˜}30 nm as evidenced by transmission electron microscopy (TEM) (FIG. 2e). The size is consistent with coarse-grained molecular dynamics simulation (FIG. 2b) and with dynamic light scattering (DLS) measurements, which show a Z-average hydrodynamic diameter of 32±2 nm and a polydispersity index of 0.17 (FIG. 2d).

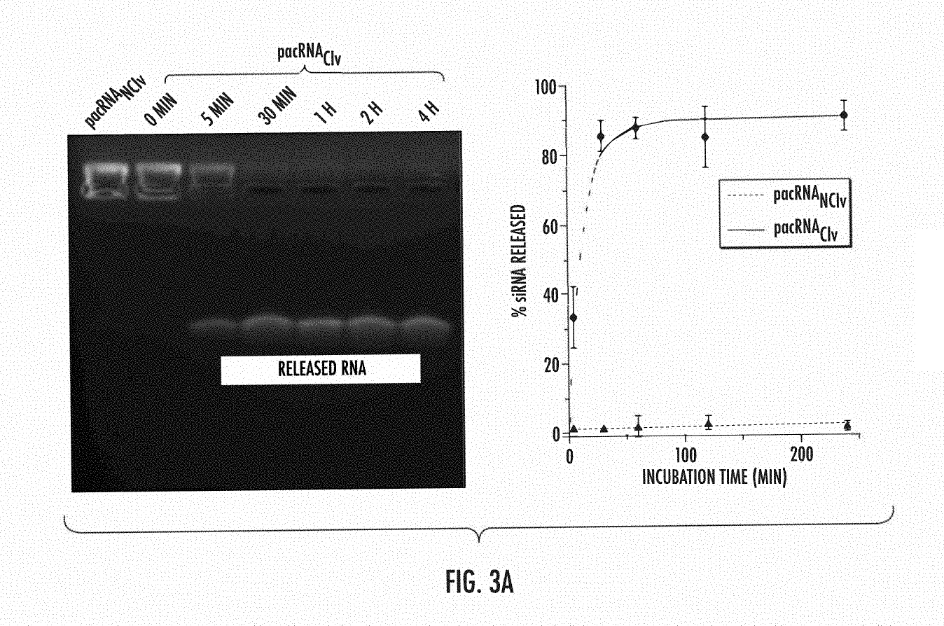

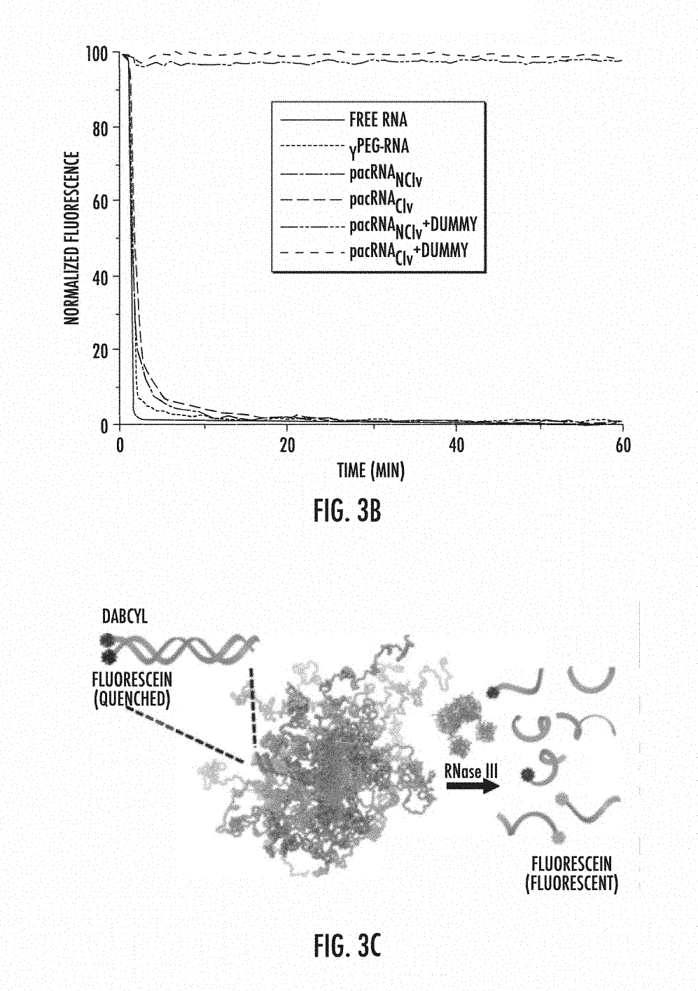

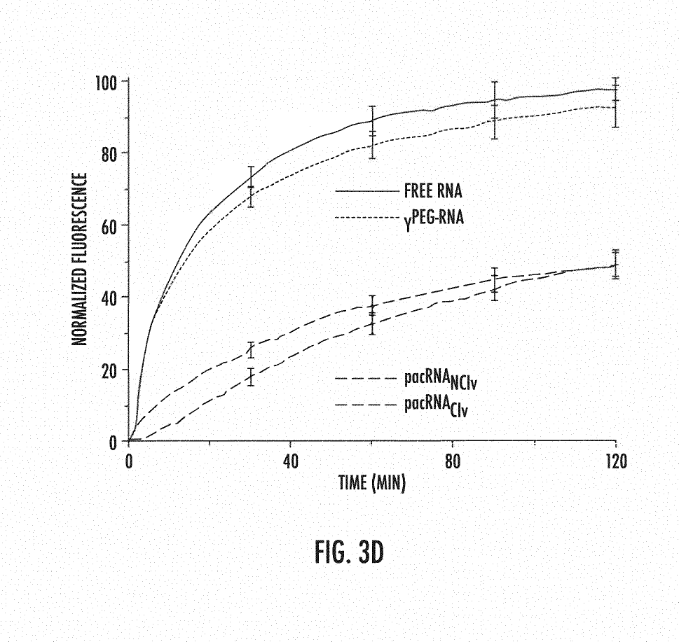

The redox-responsiveness of pacRNA_{Clv} was tested by treatment with 10 mM dithiol threitol (DTT) in phosphate-buffered saline (PBS), a condition often used to mimic the reductive intracellular environment. A time-course release profile was obtained by gel densitometry analysis of the released siRNA (FIG. 3a), which shows that ^{˜}80% of the siRNA was released after 30 min. In contrast, the stable pacRNA_{NClv} resulted in no release of the siRNA throughout the reaction. With a few exceptions, the cytoplasmic environment of tumor cells maintains a higher concentration of glutathione (GSH) than disease-free cells, and much higher than typical serum levels (^{˜}1 mM). Therefore, the difference in GSH concentration may be a contributing mechanism for passive tumor targeting, in addition to the EPR effect.

### Example 3

### Hybridization of pacRNA

Analyses of whether the pacRNAs remain capable of hybridization with a complementary (sense) sequence, and whether the elevated PEG density allows the pacRNA to resist nuclease degradation were undertaken. Hybridization and nuclease degradation are monitored by a fluorescence quenching assay, in which a quencher (dabcyl)-modified sense strand is mixed with fluorescein-labeled pacRNA containing the antisense strand. Upon hybridization, the fluorophore-quencher pair is brought to proximity, resulting in a reduction in the fluorescence signals. Degradation, on the other hand, results in the release of the fluorophore and an increase in fluorescent signal. The rates of fluorescence loss and gain are therefore indicators of the hybridization and nuclease degradation kinetics, respectively. As shown in FIG. 3b, both pacRNAs hybridized with the sense strand rapidly, with negligible difference compared with free RNA. When a scrambled sequence was added, there was no change in the fluorescent signals, which rules out nonspecific interactions. To examine the extent of nuclease resistance, RNase III (an endoribonuclease specific for dsRNA) was added to prehybridized fluorophore/quencher-bearing duplexes (0.4 unit/mL). The pacRNAs exhibited significantly prolonged nuclease half-lives (t_{0.5}: 120±4 min) compared with that of naked dsRNA (t_{0.5}:11.0±0.5 min). In contrast, a Y-shaped PEG (40 kDa)-siRNA conjugate (an architectural control) only exhibited slightly increased half-life (t_{0.5}:13±2.5 min). (FIGS. 3c-d).

### Example 4

### Cellular Uptake, Intracellular Release, Gene Regulation, and In Vitro Biocompatibility

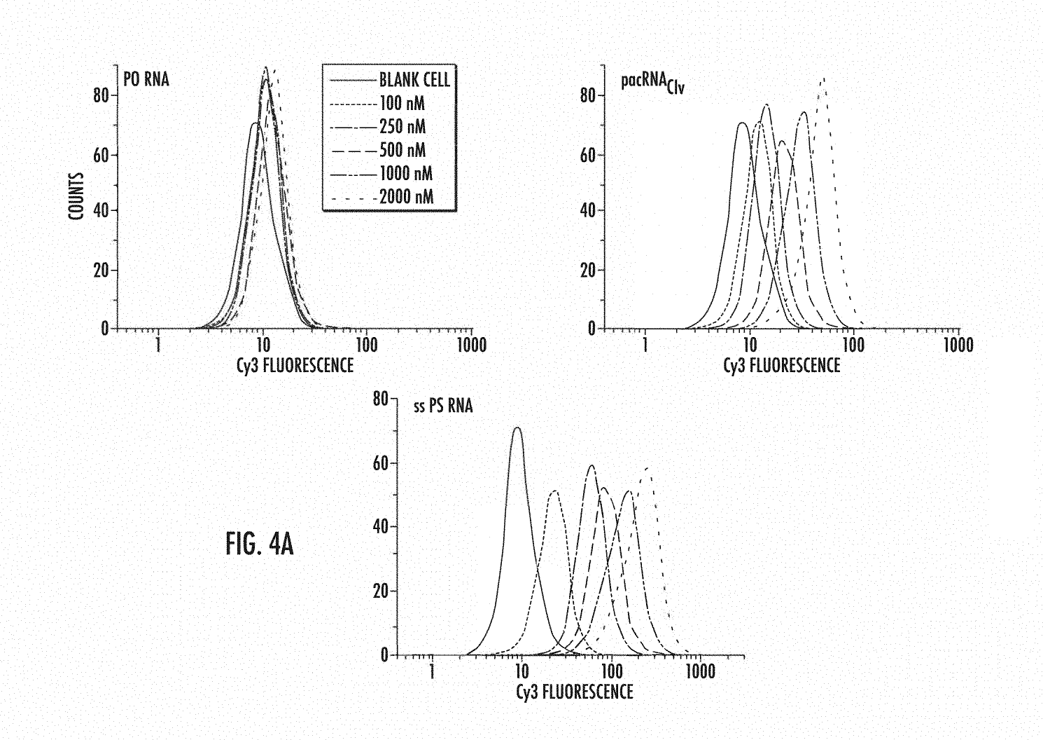

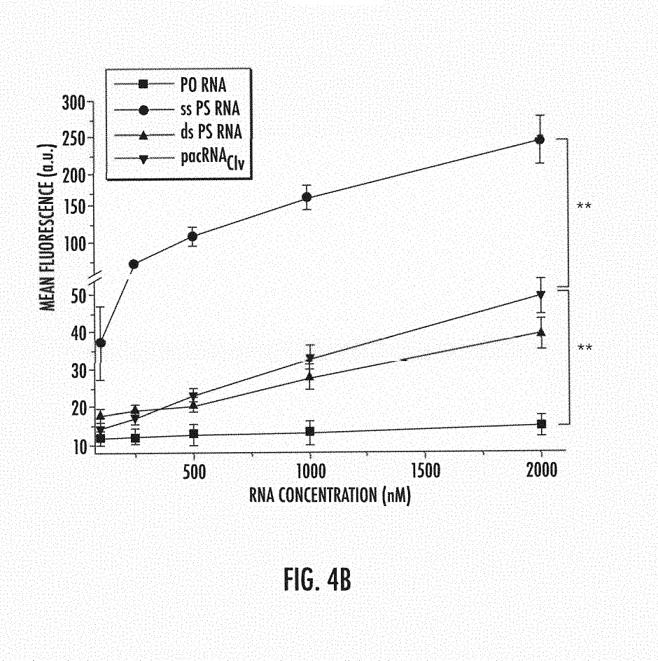

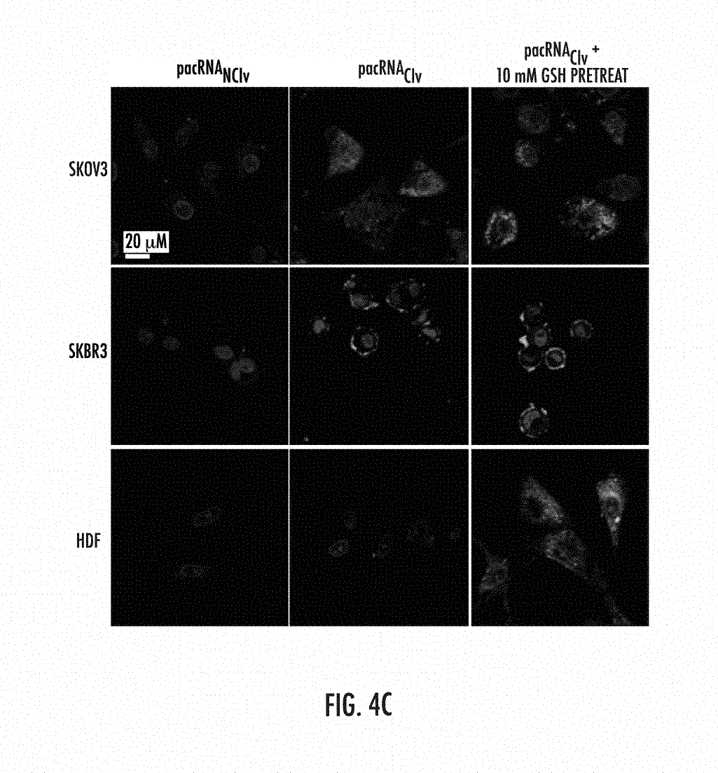

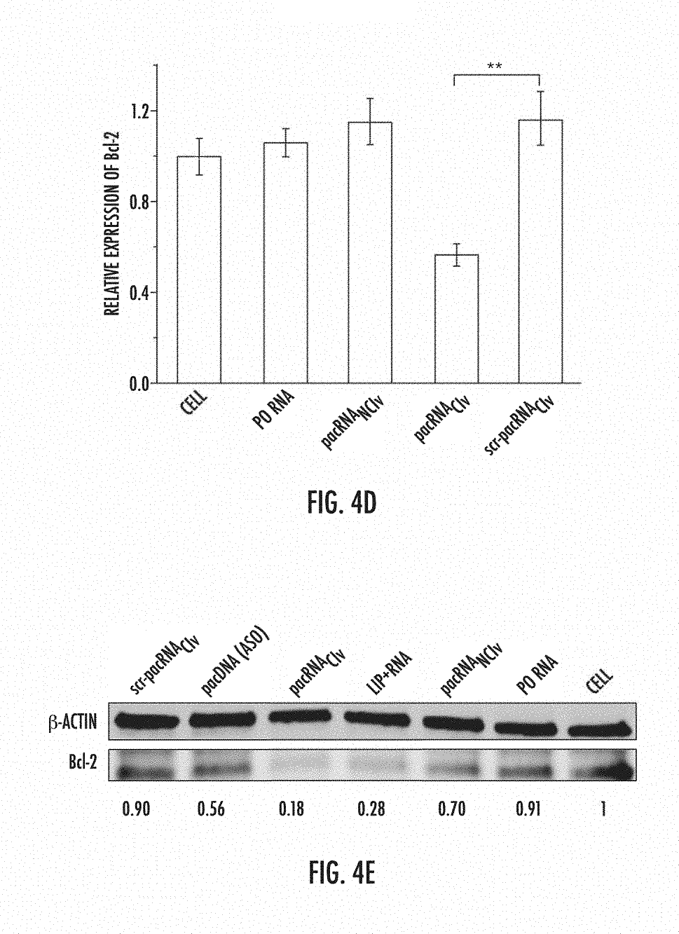

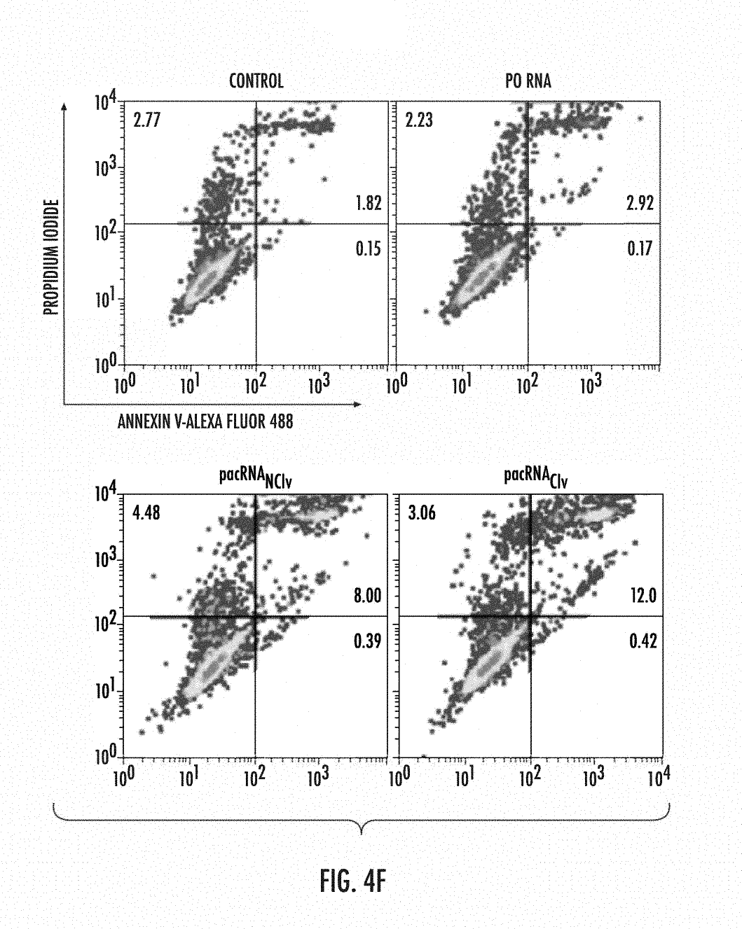

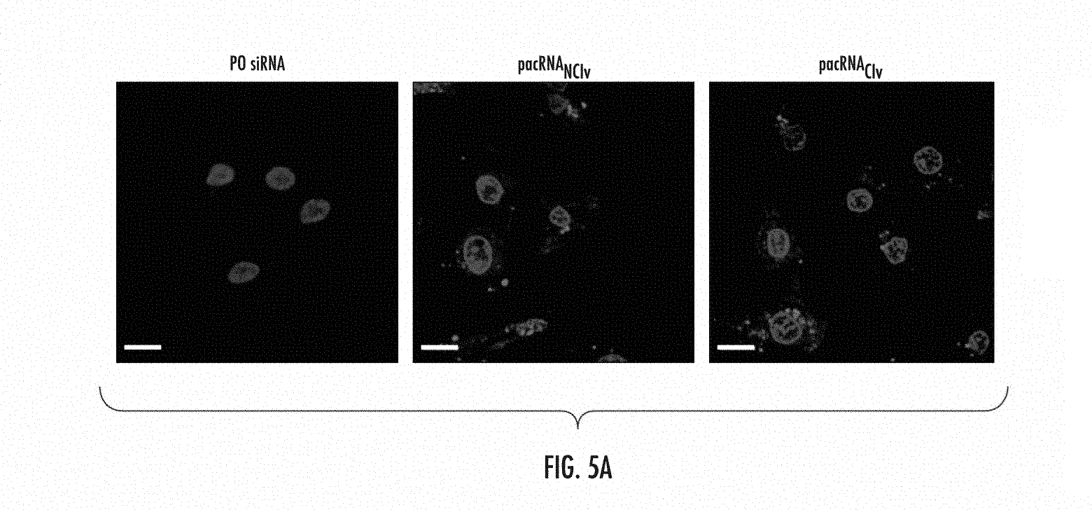

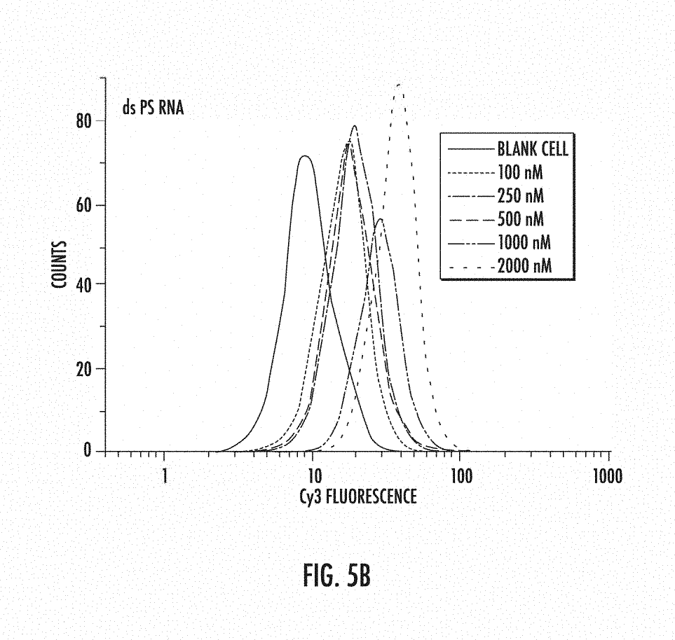

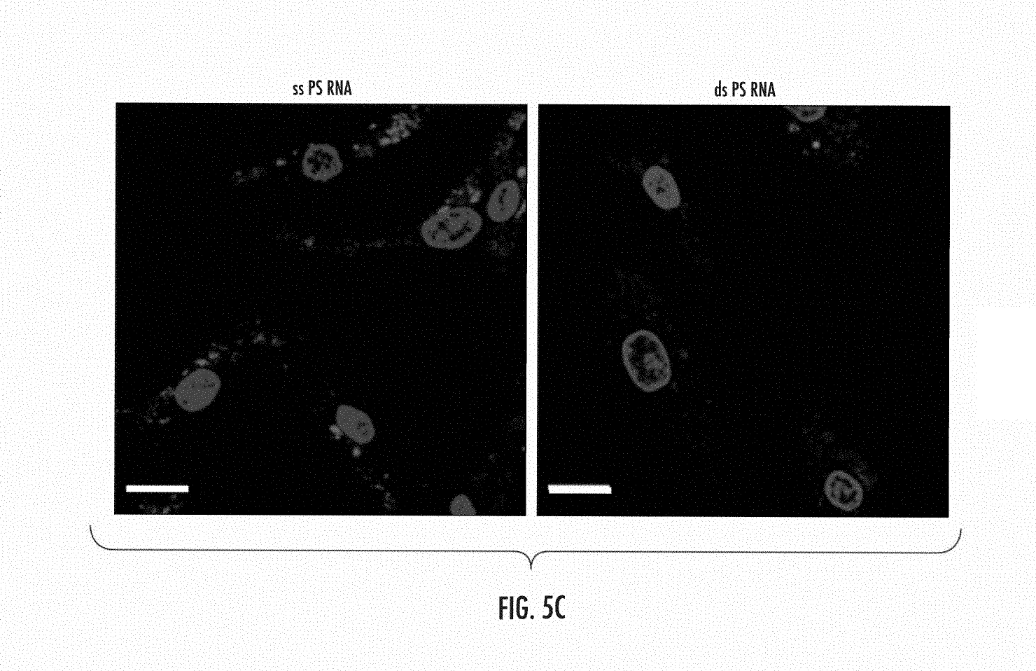

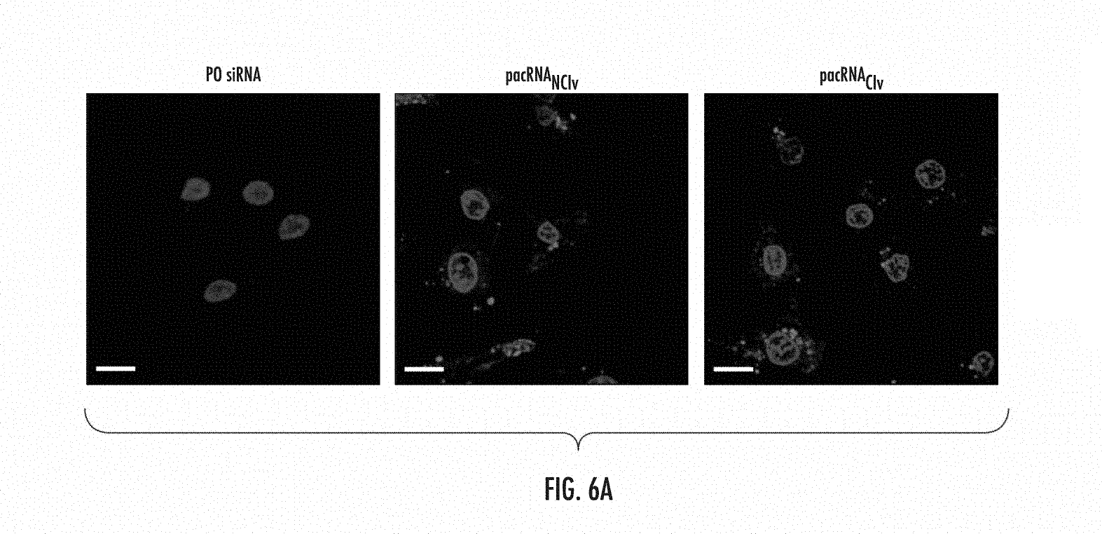

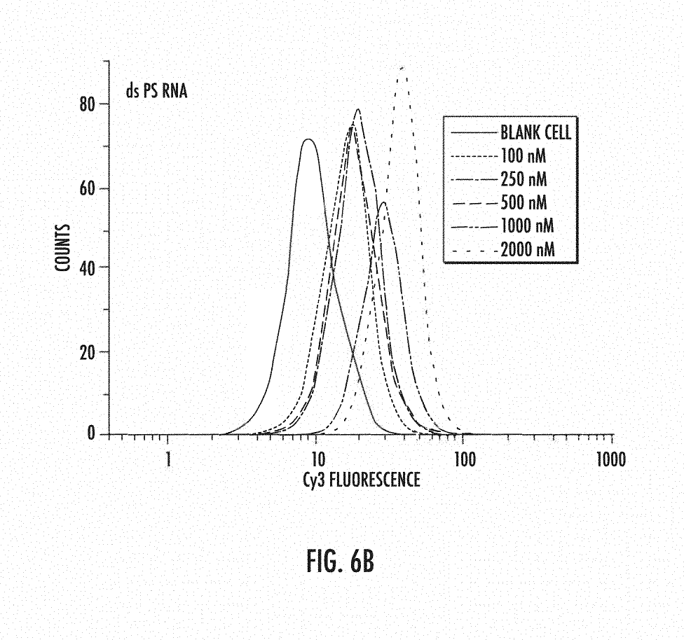

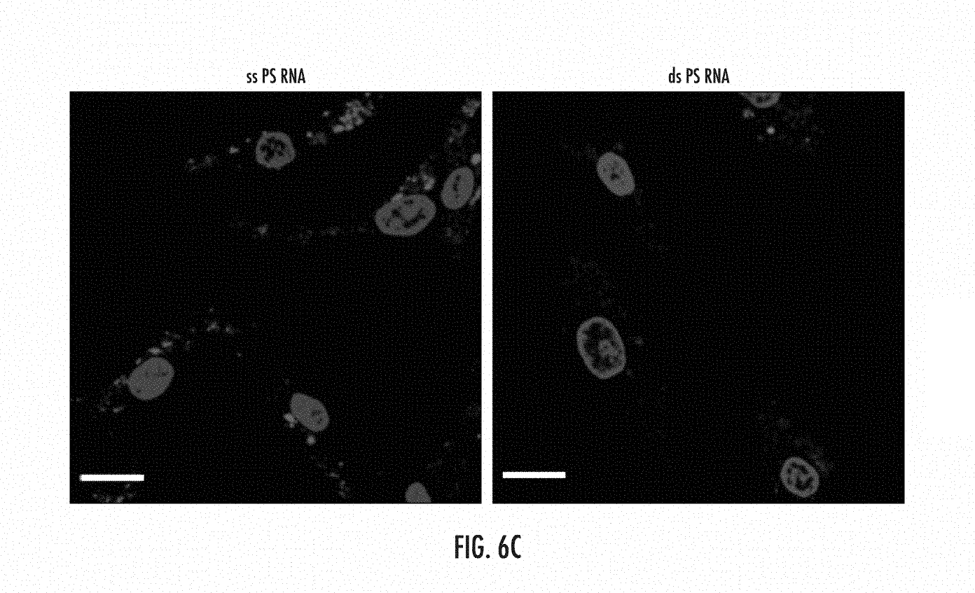

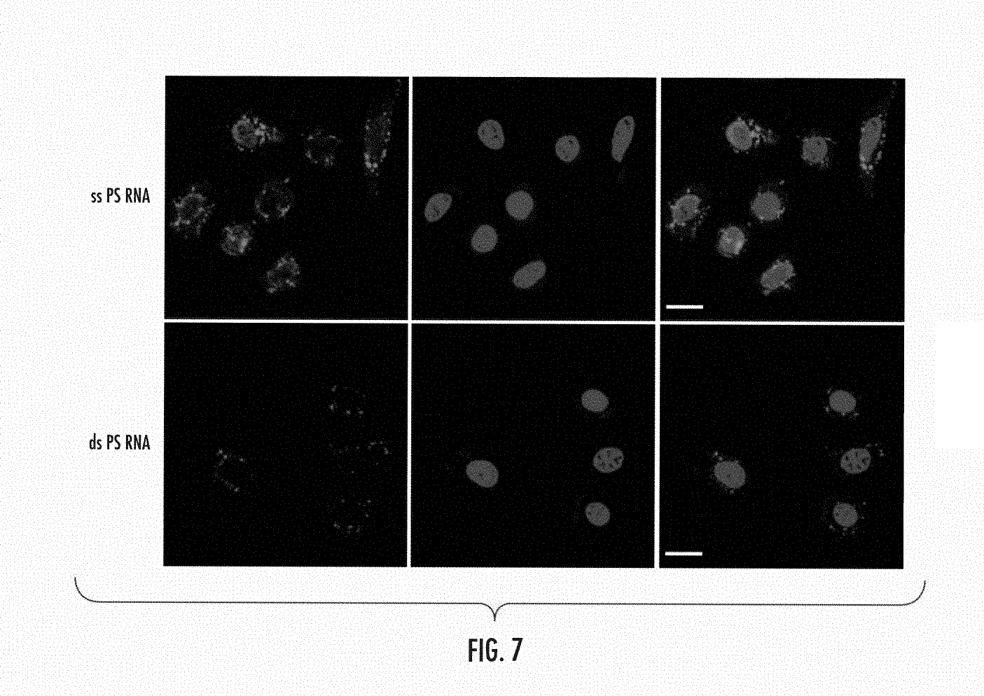

Naked PO nucleic acids undergo insignificant cellular uptake, which may occur via non-receptor-mediated endocytosis (i.e. fluid-phase or adsorptive). The somewhat “non-sticky” character of nucleic acids is one of the reasons necessitating polycationic carrier systems, which enhance cellular delivery and subsequence release from endosomes to the cytosol. Certain chemically modified nucleic acids such as phosphorothioates, on the other hand, are promiscuous binders to serum, cell membrane, and intracellular proteins, resulting in the ultimate high uptake by cells/tissues but also limited circulation in the blood and increased potential for off-target effects. Therefore, we hypothesize that the ability to moderately enhance cellular uptake above that of free nucleic acid but still maintain an overall biological stealth character plays an important role in a long-circulating vector. To examine the cellular uptake of the pacRNAs, human ovarian carcinoma cells (SKOV3) and breast adenocarcinoma cells (SKBR3) were treated for 4 h with 0.1-2 μM Cy3-labeled conjugates, free PO RNA, and a full PS version of the RNA (both ss and ds) and were subsequently analyzed by confocal fluorescence microscopy and flow cytometry. Consistent with prior studies, free PO siRNA exhibited negligible cell uptake. The pacRNA_{Clv}, on the other hand, showed 10× (SKOV3) and 22× (SKBR3) faster cell uptake compared to free RNA (FIG. 4a-b and FIGS. 5-7). While the increases are significant, the uptake for ss PS RNA was 60-82× faster than free RNA, which is comparable to the level of Lipofectamine-assisted delivery. Interestingly, the uptake for double stranded (ds) PS RNA was substantially slower than that of the ingle stranded (ss) version (^{˜}one tenth the rate), suggesting that the conformational freedom of ss PS RNA is important for its interaction with serum and membrane proteins and subsequent endocytosis. Confocal microscopy confirmed that in all cases the RNA or conjugates were internalized by the cell as opposed to being surface-associated (FIG. 5). We speculate that the pacRNA enters the cell via a non-receptor-mediated endocytotic process similar to that of naked PO RNA and free PEG, but the near-neutral surface charge of the conjugate improves the transient particle-cell interactions, thereby allowing for improved uptake.

To investigate whether the internalized pacRNA can release the siRNA payload in tumor cells, a fluorescence off-on assay using fluorescein-labeled siRNA conjugated to quencher (dabcyl)-modified bottlebrush polymer was designed. The turn-on of fluorescence is indicative of siRNA release (FIG. 4c). When tumor cells (SKOV3 and SKBR3) were treated with pacRNA_{Clv}, apparent fluorescence was observed by confocal microscopy, from mainly within compartmentalized vesicles, while only very weak signals were detected in normal cells (HDF, primary human dermal fibroblasts) under identical imaging settings. The result agrees with prior findings that the levels of intracellular GSH in certain tumor cells including SKOV3 and SKBR3 are several times higher than that in normal cells, and that the disulfide bond-reducing activity can occur within the endocytotic vesicles. In contrast, the stable pacRNA_{NClv} exhibited little fluorescence in both tumor and normal cells, indicating that the kinetics of intracellular enzymatic cleavage of the siRNA is much slower compared with the bioreductive cleavage of the disulfide bond. For control, all cells were pre-incubated with 10 mm GSH monoester (GSH-OEt) for 2 h to enhance the intracellular GSH level, followed by incubation with pacRNA_{Clv}. With the pre-treatment, fluorescence signals in all cells appear higher, and HDF cells in particular showed similar fluorescence levels to cancer cells. This observation provides direct evidence of bioreductive siRNA release from the pacRNA_{Clv}, which is important for subsequent siRNA loading into the RISC (a critical step in RNAi).

### Example 5

### RNAi Efficacy In Vitro

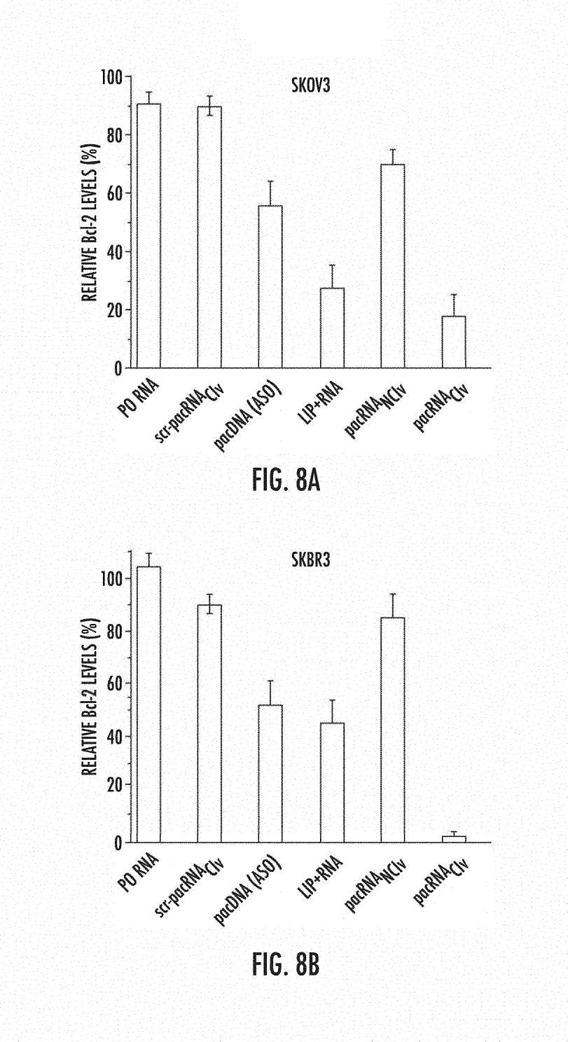

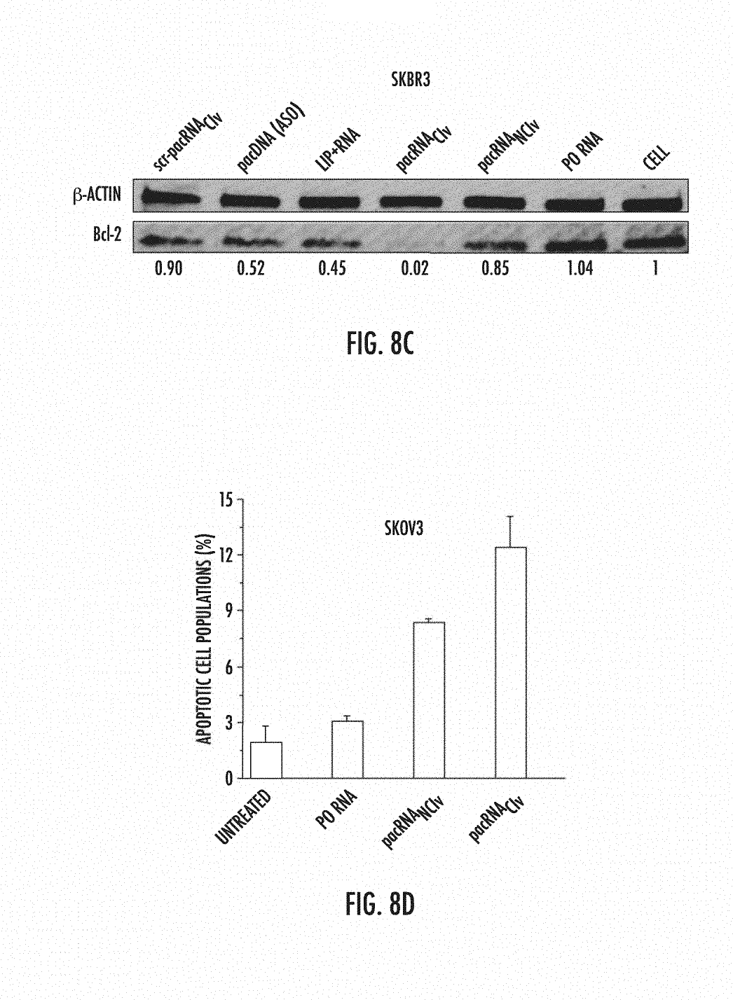

To evaluate the RNAi efficacy in vitro, SKOV3 cells were incubated with pacRNA containing a Bcl-2 siRNA sequence (Table 1) and controls. Quantitative real-time polymerase chain reaction (qRT-PCR) showed that the Bcl-2 transcript level was reduced by 43% with pacRNA_{Clv} treatment but remained nearly unchanged with the scrambled pacRNA_{Clv} and free siRNA controls (FIG. 4d). Western blotting confirmed that the pacRNA_{Clv} was the most effective in RNAi, with ˜82% knockdown by band densitometry analysis, more effective than Lipofectamine-based transfection (72%). In comparison, an ASO-based pacDNA control and the non-cleavable pacRNA_{NClv} reduced Bcl-2 expression by 44% and 30%, respectively (FIGS. 4e and 8a). The same experiments were also performed with SKBR3 cells, and a similar general trend was observed (FIG. 8b-c). Decreased Bcl-2 expression has been observed to correlate with an increase in the levels of executioner proteins (Bax and Bak) in the cytoplasm, which facilitate the permeabilization of the mitochondria outer membrane and activation of the intrinsic apoptotic pathway. Apoptosis of SKOV3 cells treated with pacRNAs and controls was monitored by FITC-annexin V/propidium iodide (PI) staining. Treatment with pacRNA_{Clv} resulted in the highest induction of apoptosis (12.42%), with the majority of the apoptosing cells in the late phase. The stable conjugate, pacRNA_{NClv}, also resulted in 8.39% of apoptotic cells (8.00% in the late phase), despite lower potency in gene silencing (FIGS. 4f, 8d). In contrast, treatment with free siRNA did not result in an appreciable number of apoptotic cells beyond the untreated cells.

TABLE 1
Oligonucleotide sequences

| | |
|---|---|
| Amine-modified | 5′-NH2-CAG CUU AUA AUG GAU |
| Bcl-2 antisense | GUA C-dTdT-3′ |
| | (SEQ ID NO.: 1) |
| DBCO-modified | 5′-DBCO-CAG CUU AUA AUG GAU |
| Bcl-2 antisense | GUA C-dTdT-3′ |
| | (SEQ ID NO.: 2) |
| Cy3-labeled and | 5′-DBCO-CAG CUU AUA AUG GAU |
| DBCO-modified | GUA C-dTdT-Cy3-3′ |
| Bcl-2 antisense | (SEQ ID NO.: 3) |
| Cy3-labeled and | 5′-NH2-CAG CUU AUA AUG GAU |
| amine-modified | GUA C-dTdT-Cy3-3′ |
| Bcl-2 antisense | (SEQ ID NO.: 4) |
| Dabcyl-labeled | 5′-Dabcyl-GUA CAU CCA UUA |
| Bcl-2 sense | UAA GCU G-dTdT-3′ |
| | (SEQ ID NO.: 5) |
| Dabcyl-labeled | 5′-Dabcyl-AUU ACA UAU ACG |
| scrambled | CCG UUG A-dTdT-3′ |
| sequence | (SEQ ID NO.: 6) |
| Scrambled | 5′-NH2-AGU AGA GCG UCA UUA |
| Bcl-2 antisense | UUA C-dTdT-3′ |
| | (SEQ ID NO.: 7) |
| Scrambled | 5′-NH2-GUA AUA AUG ACG CUC |
| Bcl-2 sense | UAC U-dTdT-3′ |
| | (SEQ ID NO.: 8) |
| Cy3-labeled PS | 5′-rC*rA*rG*rC*rU*rU*rA*rU* |
| RNA antisense | rA*rA*rU*rG*rG*rA*rU*rG*rU* |
| | rA*rC-Cy3-3′ |
| | (SEQ ID NO.: 9) |
| Dabcyl-labeled | 5′-Dabcyl-rG*rU*rA*rC*rA*rU* |
| PS RNA sense | rC*rC*rA*rU*rU*rA*rU*rA*rA* |
| | rG*rC*rU*rG-3′ |
| | (SEQ ID NO.: 10) |
| Cy5-labeled PS | 5′-rC*rA*rG*rC*rU*rU*rA*rU* |
| RNA antisense | rA*rA*rU*rG*rG*rA*rU*rG*rU* |
| | rA*rC Cy5-3′ |
| | (SEQ ID NO.: 11) |
| PS RNA sense | 5′-rG*rU*rA*rC*rA*rU*rC*rC* |
| | rA*rU*rU*rA*rU*rA*rA*rG*rC* |
| | rU*rG-3′ |
| | (SEQ ID NO.: 12) |
| DNA Bcl-2 | 5′-DBCO-TTT TCT CCC AGC GTG |
| antisense strand | CGC CAT-3′ |
| | (SEQ ID NO.: 13) |

### Example 6

### Cytotoxicity

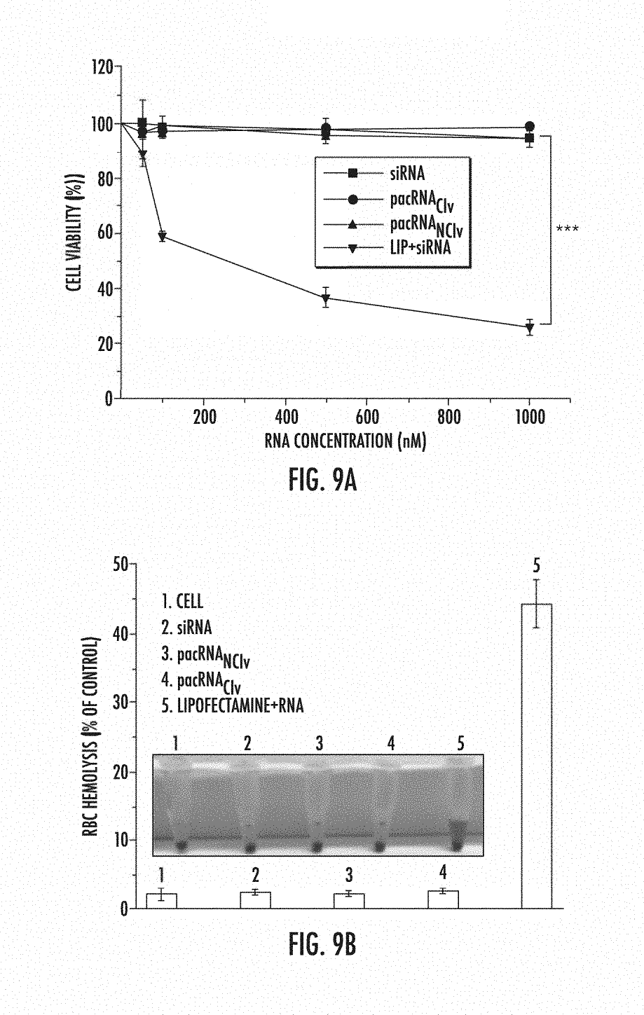

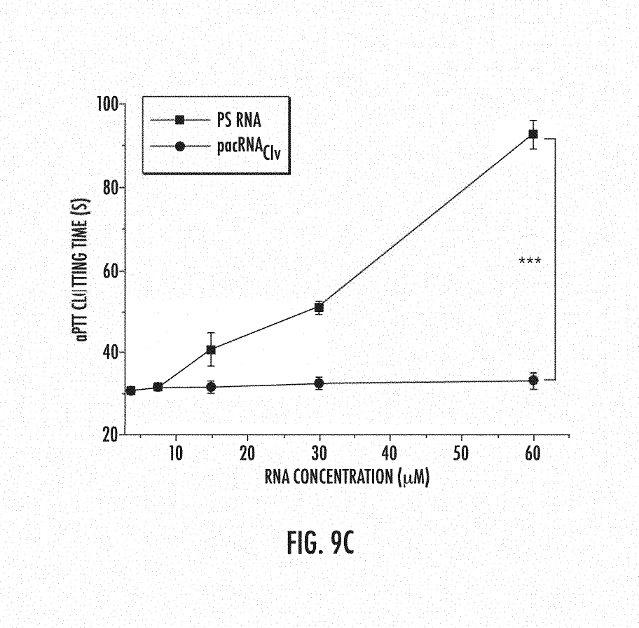

A key advantage of the pacRNA is the realization of vector-like properties using almost only PEG and native RNA, relying on the steric selectivity resulting from the unique architecture of the bottlebrush polymer. As such, pacRNAs are substantially free of the typical drawbacks associated with cationic carriers and chemically modified nucleic acids. Indeed, 3-(4,5-dimethyl-thiazol-2-yl)-2,5-diphenyl tetrazolium bromide (MTT) cytotoxicity assay indicated no toxicity for pacRNA-treated SKOV3 cells up to 1 μM of RNA (highest concentration tested), whereas Lipofectamine (cationic lipids) exhibited a half maximal inhibitory concentration (IC₅₀) of 270 nM, as expected from a typical polycationic carrier (FIG. 9a). In addition, high positive ζ potential synthetic vectors and surfactant-like carriers (e.g. liposomes, micelles, etc.) often display varying levels of blood incompatibility, e.g. aggregation of erythrocytes, hemolytic activity, etc. The pacRNA, being slightly negatively charged and completely hydrophilic, exhibited no detectable hemolysis, as estimated by measuring the amount of the hemoglobin released from red blood cells (RBCs) treated with an equivalent of 1 μ [symbol] M of RNA under physiological conditions. For comparison, Triton X-100 (a nonionic PEG-based surfactant, 1% v/v) and Lipofectamine resulted in 100% and 44% hemolysis, respectively (FIG. 9b). Another benefit of the pacRNA is the ability to inhibit protein association in a complex biological environment, which reduces the likelihood of unwanted side effects. As a demonstration, we examined the anticoagulation properties of pacRNA vs. PS RNA in human plasma (FIG. 9c). PS sequences exhibit strong non-specific interactions with serum proteins such as thrombin, resulting in pronounced prolongation of activated partial thromboplastin time (aPTT), which leads to increased risk of bleeding in patients. Indeed, the PS RNA (60 μM) showed marked anticoagulation activities, raising the aPTT by ^{˜}3s. In contrast, the pacRNAs at equal RNA concentrations resulted in only slight changes (<5%) in clotting times, indicating that the pacRNA does not disrupt the normal functions of proteins involved in the blood coagulation pathway.

### Example 7

### Pharmacokinetics, Biodistribution, In Vivo Antitumor Efficacy, and Safety

One main mechanism for anticancer nanomedicine systems to reach the pathological site is through blood circulation and extravasation via compromised vasculature, followed by intratumoral retention. Therefore, the dosage requirements for achieving high enough tumor concentration of the nanomedicine strongly depend on the longevity of the drug in blood circulation. To evaluate the plasma pharmacokinetics (PK) of the pacRNA, immunocompetent C57BL/6 mice were injected in the tail vein with free pacRNAs (both pacRNA_{NClv} and pacRNA_{Clv}), brush polymer lacking an RNA component, and naked siRNA (both PO or PS) at equal RNA/polymer concentrations. Blood samples at various predetermined time points up to 24 h were collected and analyzed (FIG. 7a). All samples rapidly distributed into tissues with distribution half-lives (t_{1/2α})≤30 min, but bottlebrush-containing samples showed much longer elimination half-lives (t_{1/2β}^{˜}13-20 h) compared with free PS siRNA (t_{1/2β}=53 min) or PO siRNA (t_{1/2β}=34 min). There were also vast differences in plasma concentration. At 1 h post injection, there was only ^{˜}0.6% of the injected free PO siRNA remaining in the plasma. While the PS siRNA exhibited significantly longer blood retention (^{˜}7.0% at 1 h) likely due to binding with plasma proteins and therefore reduction in the glomerular filtration and urinary excretion, the bottle brush polymers were much more effective in blood retention, with >47% of the injected dose remaining in circulation at 1 h and 20.6% at 24 h. The pacRNAs exhibited similarly improved blood availability (^{˜}15% at 24 h), suggesting that the brush polymer can evade renal clearance and impart its biological stealth character to the siRNA. The vast differences in blood concentration and elimination rates result in substantially elevated blood availability of the pacRNAs compared with naked siRNA, with or without the phosphorothioate backbone (AUC_{pacRNA(Clv)},∞/AUC_{PO RNA},∞=^{˜}19). All pharmacokinetic parameters are summarized in Table 2.

TABLE 2
Plasma pharmacokinetic parameters in C57BL/6 mice.

| Sample | t½ (α)(h) | t½ (β)(h) | AUC∞ (nmol/mL × h) |
|---|---|---|---|
| ds PO siRNA | 0.28 | 0.58 | 2.8 |
| ds PS siRNA | 0.26 | 0.89 | 4.1 |
| pacRNA_{NClv} | 0.28 | 13.3 | 53.6 |
| pacRNA_{Clv} | 0.30 | 14.8 | 52.0 |
| Brush polymer | 0.25 | 20.1 | 88.6 |

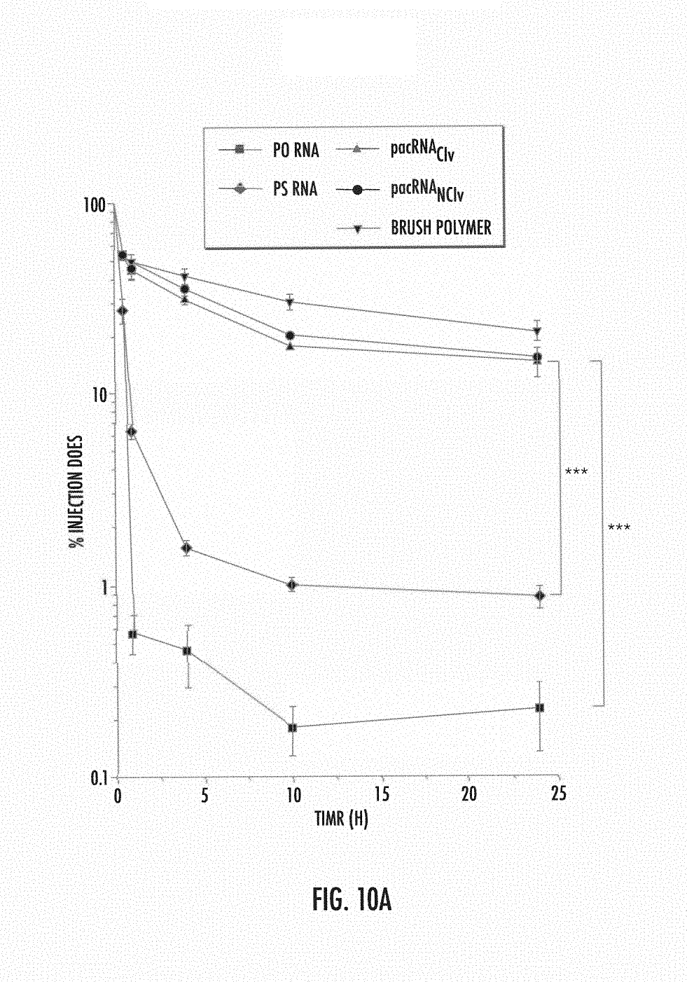

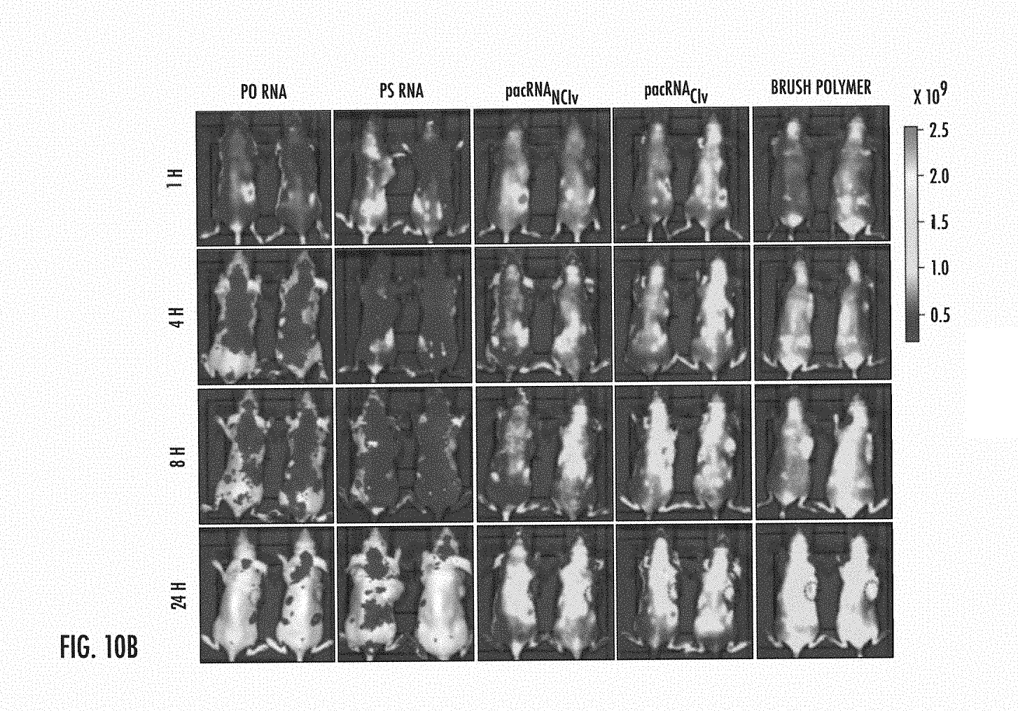

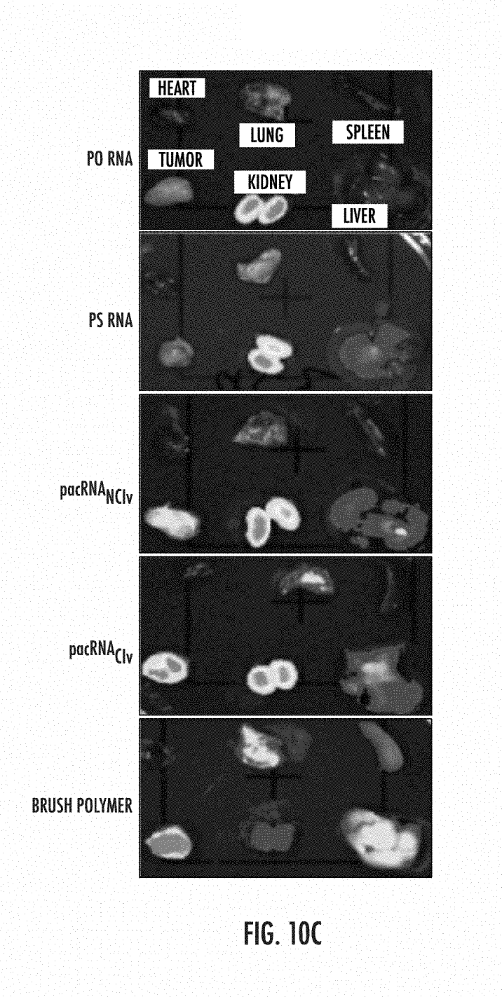

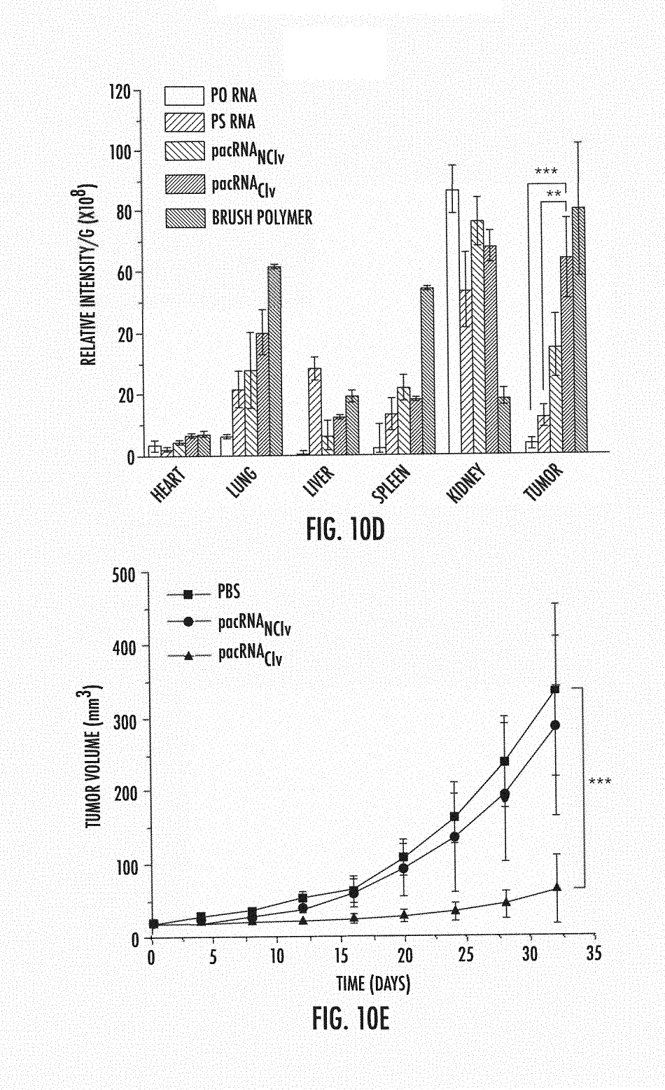

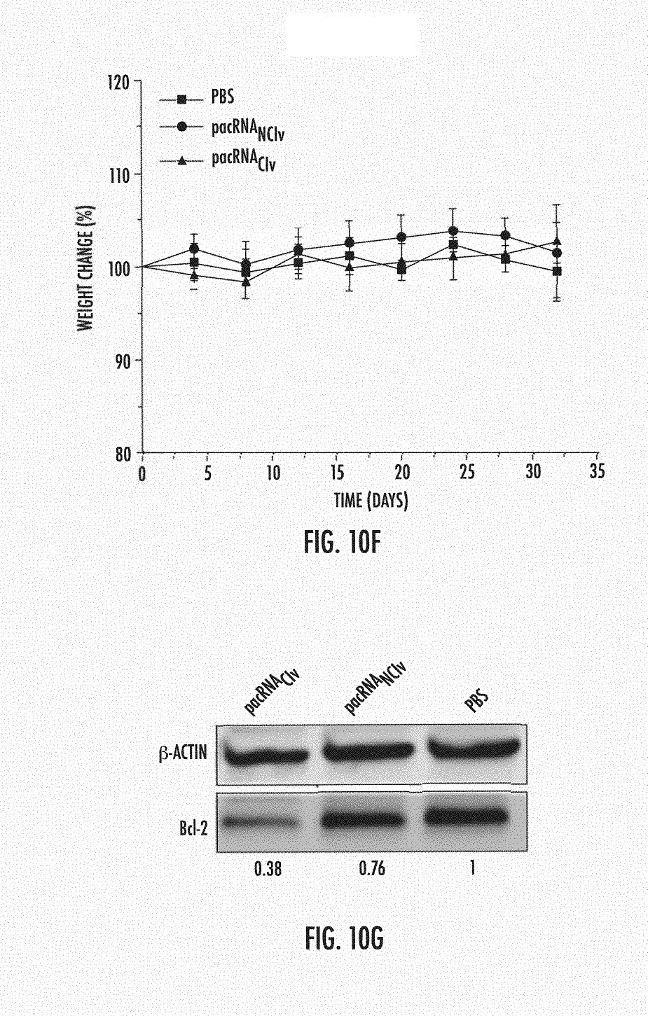

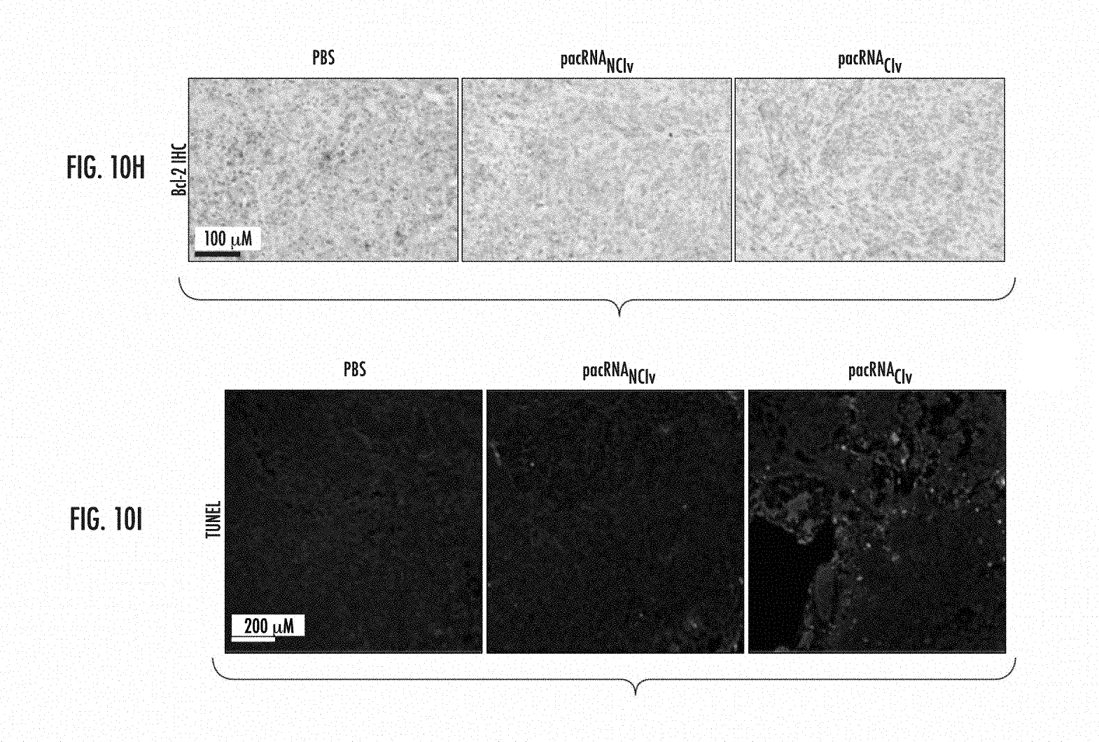

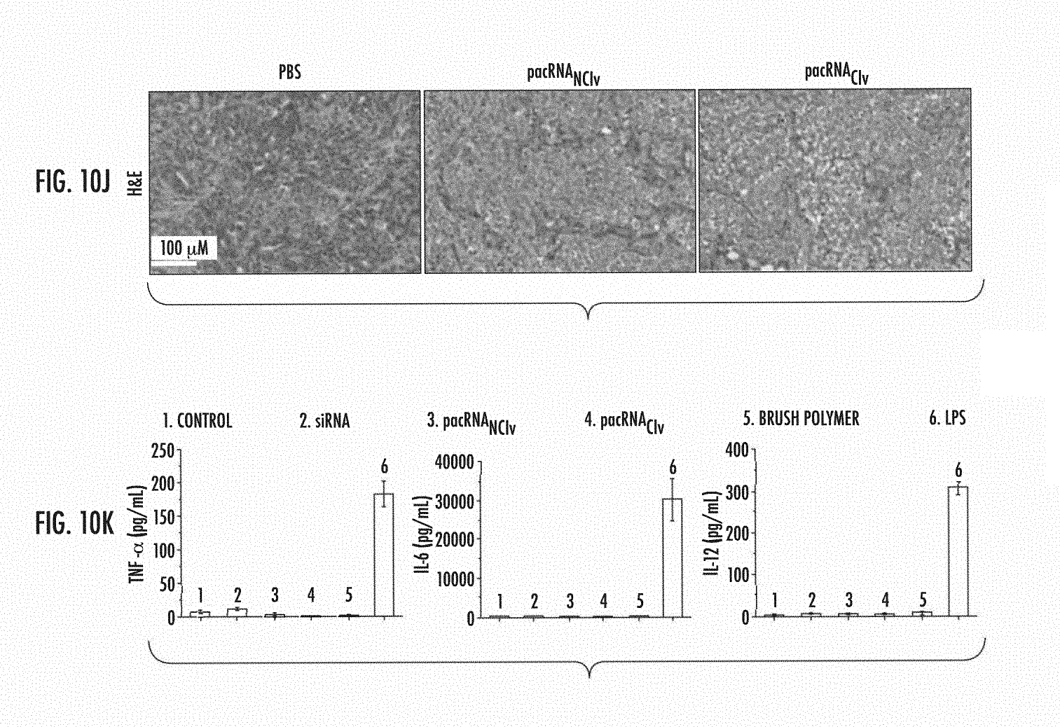

The improved pharmacokinetics of pacRNA greatly enhanced siRNA accumulation at subcutaneously inoculated SKOV3 tumor sites in BALB/c mice, likely via the EPR effect. Fluorescence imaging of both live animals and the dissected organs 24 h post injection suggests that free PO siRNA was quickly and primarily cleared by the kidney, while the PS siRNA rapidly accumulated in the liver as well as the kidney (FIG. 10b-c). Tumor uptake was minor or unobservable for the PS or PO siRNA-treated mice, respectively. Strikingly, the bottlebrush polymer exhibited the highest abundance in the tumor, followed by lung, spleen, and liver (FIG. 10d), suggesting effective tumor targeting. The tumor levels for pacRNA_{Clv} and pacRNA_{NClv} are 80% and 44% relative to the free brush, respectively, indicating that the siRNA is not completely shielded by the brush. Once cleaved, the fragments are subject to rapid renal clearance. Indeed, the ratio of tumor vs. kidney uptake (as determined by mean fluorescence/g of tissue) is 4.3 for the free brush, 1.0 for pacRNA_{Clv}, and 0.5 for pacRNA_{NClv}. Of note, the fluorescent tag is located at the outer periphery of the siRNA component on the pacRNA, and therefore cleavage at any position would cause the release of the fluorophore. The pacRNA_{Clv}, having an additional bioreductive cleavage mechanism compared with the enzyme-only pacRNA_{Clv}, accumulate more in the tumor despite a greater chance of releasing the siRNA. It is possible that the pacRNA_{NClv} primarily liberates fragments of the siRNA due to enzymatic cleavage while in blood circulation. The pacRNA_{Clv}, on the other hand, releases the siRNA more rapidly via bioreductive cleavage at the tumor site, which overpowers enzymatic cleavage, causing additional tumoral retention of the siRNA. To further examine tumor penetration depths, tumors were sliced, stained with DAPI (4′,6-diamidino-2-phenylindole), and imaged by confocal fluorescence microscopy. Appreciable fluorescence was detected in the tumors for pacRNA- and brush polymer-treated mice, while free siRNA resulted in no detectable fluorescence presumably due to poor pharmacokinetics. Notably, RNA fluorescence can be observed throughout the tumor section, including the center (^{˜}2.5 mm depth), suggesting enhanced penetration. (Data not shown).

### Example 8

### Anti-Ttumor Efficacy

To further investigate the anti-tumor efficacy of the pacRNAs, an SKOV3 xenograft model was established in athymic nude mice (BALB/c). When the xenograft reaches a volume of ca. 20 mm³, pacRNA_{Clv}, pacRNA_{NClv}, or vehicle (PBS) were administered in the tail vein (0.5 μmol/kg) once every 4th for a total of 8 doses. By day 32, the average tumor volume in the vehicle-treated 01 group had progressed to ˜334 mm³, while the pacRNA_{NClv}- and pacRNA_{Clv}-treated groups 02 exhibited reduced tumor growth, averaging at ˜284 mm3 and ˜62 mm3, respectively (FIG. 10e).

Suppression of Bcl-2 has been shown to inhibit tumor growth in several mouse xenograft models including H146, H1963, SCLC, and SKOV3, increase sensitivity to anticancer drugs, and enhance survival. Mice receiving pacRNA_{Clv} showed the most significant down-regulation of Bcl-2 protein expression (62% knockdown) following treatment, as evidenced by western blotting of homogenized tumor tissues (FIG. 10g). Immunohistostaining of tumor sections using anti-Bcl-2 08 antibodies revealed significantly reduced Bcl-2 immunoreactivity (brown deposits) in pacRNA_{Clv}- treated tumors compared with pacRNA_{NClv}- and vehicle-treated groups (FIG. 10h). Furthermore, terminal deoxynucleotidyl transferase dUTP nick end labeling (TUNEL) showed a much greater abundance of apoptotic cells for the pacRNA_{Clv}-treated group compared to control groups (FIG. 10i). Histologic apoptosis hallmarks (shrinkage of nucleus, formation of apoptotic bodies, decreased cellularity) was observed for the pacRNA_{Clv}-treated group by hematoxylin-eosin (H&E) staining of tumors sections, while the vehicle-treated group exhibited typical tumor histology, including pleomorphism and high nuclei:cytoplasm ratio (FIG. 10j). Taken together, these results strongly support the pacRNA_{Clv}, as an effective long-circulating vector for in vivo gene silencing.

### Example 9

### Safety Analysis

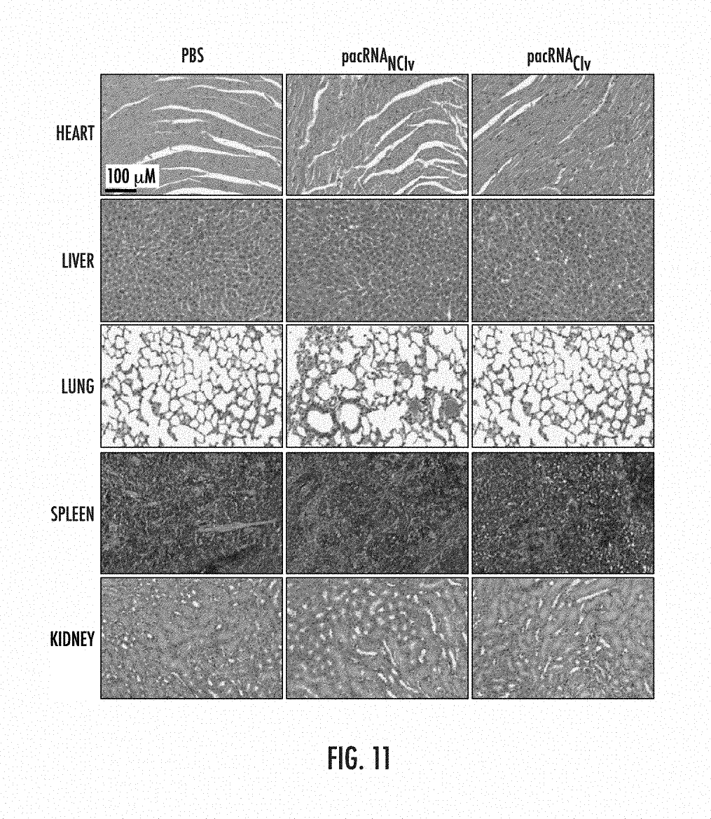

An initial in vivo safety analysis of the pacRNA was performed. Due to the benign chemical composition of the pacRNA, typical toxic and immunogenic side effects are not expected. Indeed, throughout the 32-day treatment period, mice body weight for all treatment and control groups remained constant and no obvious changes in behavior (refusal to eat, startle response, etc.) were observed (FIG. 10f). Furthermore, H&E staining of tissues obtained from major organs (including heart, spleen, liver, lung, and kidneys) in pacRNA-treated groups showed no histological variations from those of the vehicle-treated control group (FIG. 11). Nonspecific immunoactivation by RNA, pacRNAs, and the bottlebrush polymer was also investigated in C57BL/6 mice after i.v. injection by monitoring the release of cytokines related to the innate and adaptive immunity (FIG. 10k). Enzyme-linked immunosorbent assay (ELISA) showed no changes in the levels of tumor necrosis factor-α (TNF-α), interleukin-6 (IL-6), and interleukin-12 (IL-12) in the serum of mice. In contrast, lipopolysaccharide (LPS), a positive control, exhibited very strong activation of the three cytokines. These data indicate that the pacRNA is not prone to acute toxic and immunogenic responses in mice and point to a generally safe agent for in vivo use.

What is claimed:

1. A bottlebrush poly(ethylene glycol) (PEG) polymer-RNA conjugate (pacRNA) comprising one or more RNA oligonucleotides and a PEG brush polymer comprising a plurality of PEG side chains attached to a polymer backbone, wherein the one or more RNA oligonucleotides are attached to the polymer backbone via a cleavable linkage.

2. The pacRNA of claim 1, wherein the one or more RNA oligonucleotides are double stranded RNA.

3. The pacRNA of claim 1, wherein the one or more RNA oligonucleotides are single-stranded RNA.

4. The pacRNA of claim 1, wherein the length and density of the PEG side chains of the brush polymer are sufficient to provide enhanced nuclease stability to the one or more RNA oligonucleotides via steric hindrance.

5. The pacRNA of claim 1, wherein the one or more RNA oligonucleotides are short interfering RNA (siRNA).

6. The pacRNA of claim 1, wherein the one or more RNA oligonucleotides contain no phosphorothioates.

7. The pacRNA of claim 1, wherein the cleavable linkage comprises a disulfide bond.

8. The pacRNA of claim 7, wherein the cleavable linkage is dibenzocyclooctyne-N-hydroxysuccinimidyl ester (DBCO-SS-HNS).

9. The pacRNA of claim 1, wherein the one or more RNA oligonucleotides have a sequence that is complementary to a target polynucleic acid.

10. The pacRNA of claim 1, wherein the target polynucleic acid is an mRNA encoding a gene product.

11. The pacRNA of claim 1, wherein the target nucleic acid is a gene or RNA transcript specific to a cancer cell or which is over-expressed in cancer cells.

12. The pacRNA of claim 1, wherein the PEG brush polymer comprises from 25 to 60 PEG side chains attached to the polymer backbone.

13. The pacRNA of claim 1, wherein the polymer backbone is selected from the group consisting of poly(norbornene), poly(styrene), poly(meth)acrylate, polypeptide, polyether, polyamide and polyurethane.

14. A method of inhibiting expression of a gene product encoded by a target polynucleic acid comprising contacting a cell containing the target polynucleic acid with a bottlebrush poly(ethylene glycol) (PEG) polymer-RNA conjugate (pacRNA) comprising one or more RNA oligonucleotides and a PEG brush polymer comprising a plurality of PEG side chains attached to a polymer backbone to obtain uptake of the pacRNA by the cell, wherein the one or more RNA oligonucleotides are attached to the polymer backbone via a cleavable linkage and wherein the one or more RNA oligonucleotides have a sequence that is complementary to at least a portion of the target polynucleic acid.

15. The method of claim 14, wherein the one or more RNA oligonucleotides are siRNA.

16. The method of claim 14, wherein the cell is a cancer cell.

17. The method of claim 14, wherein the cleavable linkage is disrupted by the cytoplasmic environment of the cell, thereby releasing the RNA in the cell.

18. A method for promoting cellular uptake of an RNA oligonucleotide, said method comprising contacting a pacRNA with a cell, wherein the pacRNA comprises one or more RNA oligonucleotides and a PEG brush polymer comprising a plurality of PEG side chains attached to a polymer backbone, wherein the one or more RNA oligonucleotides are attached to the polymer backbone via a cleavable linkage.

19. A composition comprising a (pacRNA) comprising one or more RNA oligonucleotides and a PEG brush polymer comprising a plurality of PEG side chains attached to a polymer backbone, wherein the one or more RNA oligonucleotides are attached to the polymer backbone via a cleavable linkage.

20. A kit comprising a bottlebrush poly(ethylene glycol) (PEG) polymer-RNA conjugate (pacRNA) comprising one or more RNA oligonucleotides and a PEG brush polymer comprising a plurality of PEG side chains attached to a polymer backbone, wherein the one or more RNA oligonucleotides are attached to thepolymer backbone via a cleavable linkage and wherein the one or more RNA oligonucleotides have a sequence that is complementary to at least a portion of a target polynucleic acid.
